(19) United States
(12) Patent Application Publication
(10) Pub. No.: US 2006/0166894 A1
(43) Pub. Date: Jul. 27, 2006

(54) Ace-inhibitors having antioxidant and no-donor activity

(75) Inventors: Abdullah Ibrahim Haj-Yehia, Neve Shalom (IL); Mohamed Amin Khan, Morgan Hill, CA (US); Bashir Ali Qadri, Kfar Nahif (IL)

Correspondence Address: MORRISON & FOERSTER LLP, 755 PAGE MILL RD, PALO ALTO, CA, 94304-1018, US

(73) Assignee: Yissum Research Development Company of the Hebrew University of Jerusalem, Jerusalem (IL)

(21) Appl. No.: 10/536,628

(22) PCT Filed: Nov. 27, 2003
(86) PCT No.: PCT/IL03/01006

§ 371 Date: Dec. 19, 2005

Related U.S. Application Data
(60) Provisional application No. 60/429,864, filed on Nov. 29, 2002.
(60) Provisional application No. 60/430,003, filed on Nov. 29, 2002.

(51) Int. Cl. A61K 38/04; A61K 31/445; A61K 31/4015; A61K 31/655; A61K 31/385; A61K 31/16

(52) U.S. Cl. 514/19; 514/149; 514/317; 514/424; 514/440; 514/616

(57) ABSTRACT

Multifonctional ACE inhibitor compounds are provided, that combine ACE-inhibiting activity with capability to scavenge superoxide and other reactive oxygen species, and that may further function as nitric oxide donors. The compounds are useful for preventing or treating various disorders, including cardiovascular, and diabetes associated disorders.

### TECHNICAL FIELD

The present invention relates to multifunctional ACE (angiotensin converting enzyme) inhibitor compounds that are capable of, in addition to inhibiting ACE, scavenging superoxide or other reactive oxygen species, and optionally also acting as NO-donors. The invention further relates to methods of using such compounds in the treatment of various pathological conditions.

### BACKGROUND OF THE INVENTION

Hypertension is a major disorder affecting the populations of developed countries. The pathology of hypertension is multifactorial and in cases of inappropriate or inadequate treatment can lead to heart disease and/or injury to organs such as the kidneys, blood vessels, eyes and other vital systems [Amery A. et al.: Lancet 1 (1985) 1349-54].

There is much evidence to support a relationship between the development and pathology of hypertension and oxidative stress—an imbalance between the production of reactive oxygen species (ROS) and the endogenous mechanisms for protecting against ROS, including antioxidant enzymes like superoxide dismutase (SOD), glutathione peroxidase, glutathione reductase, catalase and low molecular weight antioxidants like vitamin C, vitamin E and glutathione [Ames B. N. et al.: Proc. Natl. Acad. Sci. USA. 90 (1993) 7915-22].

Hypertension usually accompanies other diseases related to oxidative-stress, such as diabetes, atherosclerosis, cancer and also diseases known to be related to overproduction of ROS such as alcoholism, smoking and morbid obesity.

Recent research suggests that a direct relationship exists between hypertension and states of oxidative stress, depletion of antioxidant capacity, accelerated cell ageing and depletion of cellular energy. This theory is based on mechanisms thought to underlie the pathology of hypertension such as elevated oxidative injury, increased fibrogenesis, inhibition of Na⁺—K⁺ ATPase pump activity and cardiac hypertrophy. Existing therapy for hypertension includes vasodilators and other blood pressure reducing agents that can reduce mortality due to heart failure and slow the development of other complications of hypertension.

Angiotensin converting enzyme (ACE) inhibitors constitute a cornerstone in the treatment of hypertension and in vascular protection. The first ACE inhibitor (ACEI), captopril, was described in 1977, and other recently developed ACEI can act on the crucial enzyme that generates the potent vasoconstrictor—angiotensin II (Ag-II)—from angiotensin I (Ag-I) [Opie L. H.: Drugs for the heart, 5th ed. (2001) pp 107-15.3].

Angiotensin converting enzyme (ACE) is a peptidylcarboxypeptidase, which catalyzes the cleavage of dipeptides at the carboxy terminal. ACE is responsible for the conversion of Ag-I to Ag-II and for the deactivation of bradykinin (hence the alternative name Kininase). Ag-II is a peptide that promotes blood vessel contraction and thus blood pressure elevation. Deactivatation of bradykinin, a peptide that induces smooth muscle relaxation, is another way in which ACE is thought to elevate blood pressure. ACE inhibition is therefore vasodilatory due to the decreased formation of angiotensin II, and potentially due to the increased bradykinin activity.

Human ACE consists of 1278 amino acids, forming two homologue domains. Each homologous domain contains two main sites: catalytic and binding. The enzyme occurs in all vascular beds but it is chiefly found in the vascular endothelium of the lungs [Garison J. C. and Peach M. J.: Cardiovascular Drugs, In: Goodman and Gillman's: The Pharmacological Basis of Therapeutics, Goodman A. G., Rall T. W., Nies A. S., Taylor P. editors., 8^{th} ed. Pergamon Press, USA. p. 752 (1990)].

The structure of the enzyme was extensively studied in efforts to explain the structure-activity relationship of enzyme inhibitors isolated from the venom of Bothrops jacaraca and their synthetic analogues. In one proposed model, the enzyme is divided into two main domains: obligatory (“oblig.bind.”) and auxiliary (“aux.bind.”) (FIG. 1). Both substrates (“subst.”) and inhibitors bind to the enzyme catalytic site in the same manner, which involves attachment to a number of specific binding sites. Spectroscopic tests have shown that the binding site of the enzyme contains a zinc ion. This binding site is considered as a key target for the development of the new nonpeptide inhibitors of the ACE. The natural enzyme substrates and peptide inhibitors do not usually bind to the positively charged zinc ion, while nonpeptide inhibitors do.

According to the model proposed for ACE inhibition [Ondetti M. A.: Circulation 77 (supp I) (1988) I74-I78], the structural requirements for an effective inhibitor are a carboxylic acid or ester group at one side of the molecule, a carbonyl, or preferably, an amide group, a methyl group in an alpha position to the carbonyl group, a group that can bind to the zinc ion, and the presence of pyrrolidine in the carboxylic side chain.

Investigations into the ACE inhibitor binding site led to the synthesis of potent new non-peptide ACE inhibitors, such as Captopril and Enalapril (FIG. 2).

Binding of Ag-II to its receptor induces smooth muscle contraction via a complex signalling pathway. The pathway starts with phospholipase C stimulation causing breakdown of phosphatidylinositol bisphosphate to inositoltriphosphate (IP₃) and diacylglycerol. IP₃ liberates calcium from intracellular store, such as the sarcoplasmic reticulum, to stimulate muscular contraction and hence vasoconstriction. Diacylglycerol activates protein kinase C, which transfers phosphate from adenosine triphosphate (ATP) to a target protein leading to the stimulation of proto-oncogenes.

Activation of protein kinase C through ligation of Ag-II receptors is thought to promote ventricular hypertrophy. Furthermore, ligation of Ag-II receptors can induce the activation of NADPH oxidase via a signal transduction involving protein kinase C and other molecules. Activation of NADPH oxidase leads to the generation of superoxide anions [Griendling, K. K. et al.: Circ. Res. 74 (1994) 1141-48; Rajagopalan, S. et al.: J. Clin. Invest. 97 (1996) 1916-23]. It is believed that production of superoxide anions following activation of angiotensin II receptors contributes to the biological effects of angiotensin. Rajagopalan [Ibid.] found that angiotensin II induces elevation of blood pressure accompanied with a remarkable elevation in superoxide together with a decrease in the release of endothelial nitric oxide (NO). This increase in superoxide levels did not occur when the elevation of blood pressure was induced by norepinephrine. On the other hand, there was no elevation of the blood pressure after adding the superoxide dismutase (SOD) enzyme together with angiotensin II, while the addition of SOD to norepinephrine did not prevent the elevation in blood pressure. These results indicate that angiotensin induces elevation of blood pressure through elevation of endogenous superoxide free radicals [Rajagopalan, Ibid.]. Therefore, scavenging superoxide anions at the site of angiotensin action, could lead to a reduced response to angiotensin.

The production of free radicals in the vascular system by angiotensin II seems to have a major rule in the development of hypertension and other cardiovascular diseases related to the renin-angiotensin system. Recent studies indicate the importance of supplementary antioxidants together with smooth muscle relaxant for the treatment of hypertension in order to prevent the pathological development of hypertension and other cardiac diseases.

Supplementation of exogenous antioxidants in these conditions may prevent tissue damage and the progress of the disease but it does not seem to be a solution, as external administration of antioxidants cannot restore the antioxidant capacity in the injured tissue.

In recent published research concerning the antioxidant activity of the different available ACEI, it was found that the sulfhydryl containing ACEI have better antioxidant activity than other ACEI's, due to the ability of the thiol group to quench reactive oxygen species [Bartosz M.: Free Radical Biology and Medicine 23 (1997) 729-35; Mak I. T.: Biochem. Pharmacol. 40 (1990) 2169-75].

The endothelial derived relaxing factor nitric oxide (NO) has a great importance in regulating the circulatory system and blood pressure besides other important systems in the body. It is produced in the body by a variety of tissues such as the nervous system, muscles, liver and the immune system. NO-donors can be used clinically for the treatment of cases where depletion of NO is observed such as ischemic heart disease. Unfortunately, existing NO-donors are known to elicit development of resistance and their efficacy is limited. The major problem arises from the fact that high levels of NO together with elevated levels of superoxide may lead to the production of peroxynitrite which is another potent free radical species, and can effect severe tissue damage [Munzel T. J: Clin. Invest. 95 (1995) 187-94]. Recent evidence shows that, in vascular complications of diabetes, it is peroxynitrite rather than NO itself that is responsible for the vascular disorders. Indeed, peroxynitrite is one hundred times more potent than NO in causing some of the detrimental effects originally attributed to NO, such as inhibition of cellular respiration through inactivation of critical mitochondrial enzymes.

NO is formed from the amino acid L-arginine by several forms of NO synthases, and plays a role in a number of physiological functions, including the relaxation of airway smooth muscle. NO formed in endothelial cells in response to chemical agonists and to physical stimuli plays a key role in regulation of vascular tone, platelet aggregation and adhesion, as well as modulating smooth muscle proliferation [Haj-Yehia A. et al.: Drug. Development Res. 50 (2000) 528-36]. NO overproduction has also been associated with numerous disease states (WO 99/66918).

Publications disclosing nitric oxide donor compounds or compounds which promote the synthesis of nitric oxide include WO 98/42661, WO 99/37616, WO 00/31060, WO 97/34871, WO 00/35434, WO 99/62509, WO 97/25984, WO 00/67754, WO 9961018, WO 99/61430, WO 97/31654, WO 96/32946, WO 00/53191, U.S. Pat. Nos. 6,248,895 and 6,232,331 and Wolf et al.: J. Neurosurg. 89 (1998) 279-88. Publications disclosing nitric oxide scavenger compounds include WO 98/55453.

The endothelium, in addition to producing NO, also produces superoxide (SO) anion and other reactive oxygen species (ROS) under physiological conditions. Despite SO being a reducing agent that is itself incapable of initiating oxidative reactions, SO is considered the most important source of oxidative stress. Compounds for the removal of SO are described in the art, including WO 96/39409 and U.K. Pat. App. No. 2349385A.

Many disease states, including diabetes mellitus and various cardiovascular diseases, are associated with oxidative stress and endothelial dysfunction. Nitroglycerin (GTN) has been used for the treatment of various types of myocardial ischemia. Because of its pathogenic nature (chronicity with acute exacerbation), prophylactic and acute treatments are necessary to prevent complications with potentially fatal outcomes (>25% death for acute MI). However, the phenomenon of tolerance to the anti-anginal effects of GTN and to all other existing organic nitrates is of a special clinical significance. In particular, early development of tolerance to the drug is by far the most serious drawback of nitrate therapy.

A number of cardiovascular conditions have been recognized, (e.g., angina, hypertension, arrhythmias, congestive heart failure) and a number of other conditions (e.g., migraine, tachycardia such as sinus, pheochromocytoma, thyrotoxicosis, tension, anxiety, and the symptoms of hyperthyroidism) have been recognized, many of which have overlapping and interacting etiologies.

Various compounds and treatments for cardiovascular conditions are disclosed in the art, for example, in U.S. Pat. Nos. 6,444,702, 6,417,207, 6,255,296, 6,051,571, 6,440,961, 6,429,219, 6,423,724, and 6,248,895.

Similarly, compounds and treatments for migraines are disclosed in the art, for example, U.S. Pat. Nos. 6,458,840, 6,458, 830, 6,444,702, 6,376,550, 6,414,505, 6,403,627, 6,355,689, 6331,553, 6,265,441, 6,423,724, and 6,455,549.

Various compounds and treatments for sinus tachycardia are disclosed in the art, for example, U.S. Pat. No. 6,100,297.

Compounds and treatments for hypertension are disclosed in the art, for example, U.S. Pat. Nos. 6,440,961, 6,429,219, 6,423,724, 6,214,817, and 6,455,542.

Various compounds and treatments for the symptoms of hyperthyroidism are also disclosed in the art, for example, U.S. Pat. Nos. 6,110,959, 6,121,309, and 6,437,165.

ACE inhibitors are useful in the treatment of hypertension. Inhibition of ACE lowers systemic vascular resistance and mean, diastolic and systolic blood pressures in various hypertensive states. The effects are readily observed in animal models of renal and generic hypertension. In humans subjects with hypertension, ACE inhibitors commonly lower blood pressure (except when due to primary aldolsteronism).

ACE inhibitors alone normalize blood pressure in approximately 50% of patients with mild to moderate hypertension, and many consider ACE inhibitors first-line drugs for the treatment of high blood pressure. About 90% of patients with mild to moderate hypertension will be controlled by the combination of an ACE inhibitor with either a Ca+ channel blockers, alpha adrenergic receptor blockers or diuretic [Zusman, R. M.: Am. J. Cardiol. 72 (1993) 25H-36H].

Over the past few years, several large clinical studies have examined the usefulness of ACE inhibitors in patients suffering from left ventricular systolic dysfunction. ACE inhibitors are involved in reductions of pulmonary arterial pressure, pulmonary capillary wedge pressure, and left arterial and left ventricular filling volumes and pressure. The beneficial effects of ACE inhibitors on systolic dysfunction also involve improvements in ventricular geometry (ventricular remodeling). In heart failure, ACE inhibitors reduce ventricular dilatation and tend to restore the heart to its normal elliptical shape.

In view of the ultimate importance of the ACE inhibitor-related treatments, there is a need for new improved drugs having ACE activity. It is therefore an object of this invention to provide new ACE inhibitor compounds.

Following the observations that confirm the major role played by ROS in the development of high blood pressure, that confirm the potent relaxant activity of nitric oxide, that show beneficial effects of ACE inhibitors in the treatment of high blood pressure, and that suggest the effect of superoxide anions on angiotensin after activation of NADPH oxidase, it is a further object of this invention to provide novel ACE inhibitors with antioxidant properties.

It is another object of this invention to provide multifunctional ACE inhibitors comprising, beside ROS-scavenging activity and ACE-inhibiting activity, also NO-donating activity.

Other objects and advantages of present invention will appear as description proceeds.

### BRIEF SUMMARY OF THE INVENTION

This invention relates to multifunctional ACE inhibitor possessing, beside ACE inhibiting activity, also antioxidant activity that enables scavenging reactive oxygen species (ROS), and optionally possesses also nitric oxide (NO) donating capability.

This invention is further directed to a method for treating and preventing a disorder in which treatment with an ACE inhibitor is indicated, and mainly cardiovascular disorders, renal disorders, and diabetes associated disorders. The use of said compounds in the preparation of a medicament is further provided. Preferred disorders to be treated and prevented according to this invention comprise ischaemic heart disease, angina pectoris, myocardial infarction, congestive heart failure, cardiomyopathy, atherosclerosis or Reaven's syndrome, ischaemia-reperfusion tissue injury, peripheral vascular disease, critical limb ischaemia, palpitations, arrhythmia, tachycardia, sinus, thyrotoxicosis, pheochromocytoma, tension, anxiety, arterial aneurysm, microvascular diseases, hypertension selected from pulmonary-, systemic-, ocular-, obesity-, and pregnancy-induced, impotence, diabetes mellitus, hypercholestemia, insulin-resistance and glucose intolerance in diabetes, endothelial dysfunction-induced diseases, drug or disease induced nephropathy, and migraine.

This invention further provides a multifunctional ACE inhibitor compound comprising i) an ACE inhibitor component, ii) at least one ROS-scavenger component, and optionally iii) at least one NO-donor component. Said ACE inhibitor component may comprise, or may be derived from, compounds used in medicine as ACE inhibitors, as well as other compounds exhibiting affinity for ACE. Said multifunctional ACE inhibitor comprises a ROS-scavenger component that may be an antioxidant reacting with ROS, such as superoxide, hydroxyl radicals, peroxynitrite, and hypochlorite. A preferred ROS-scavenger component may be, for example, selected from a substituted N-oxide free radical, a substituted or unsubstituted lipoic acid moiety; examples of said NO-donor component comprise —ONO₂, —ONO, —SNO, and —NONOate. Said ACE inhibitor component may comprise, e.g., Alacepril, Benazepril, Captopril, Ceronapril, Cilazapril, Delapril, Enalapril, Enalaprilat, Fosinopril, Imidapril Lisinopril, Moveltopril, Perindopril, Quinapril, Ramipril, Spirapril, Temocapril, and Trandolapril.

In a preferred embodiment of this invention, a multifunctional ACE inhibitor has Formula I 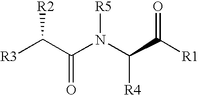
where R¹ may be selected from hydrogen (O), hydroxyl (OH), amino (NH₂), and alkoxy; R² may be selected from H and lower alkyl; R³ may be selected from -alkylene-Y and Y, wherein Y is a radical selected from the group consisting of: 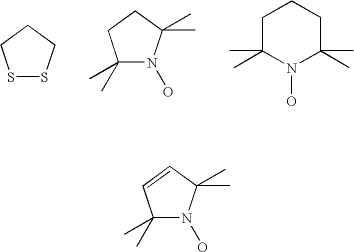
R⁴ may be lower alkyl or H; R⁵ may be selected from H, lower alkyl, -alkylene-Y or Y, wherein Y is a radical selected from the group consisting of: 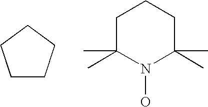
or R⁴ and R⁵ together form a group selected from the formulae: 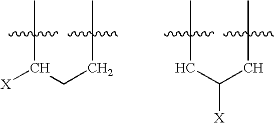
wherein X is selected from H, OH, SH, NH₂, ONO₂, SNO and NONOate.

In another preferred embodiment of this invention, a multifunctional ACE inhibitor has Formula II 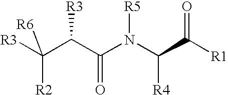
where R¹ may be selected from H, OH, NH₂, and alkoxy; R² may be independently selected from SH, SNO; R³ may be selected from -alkylene-Y and Y, wherein Y is a radical selected from the group consisting of: 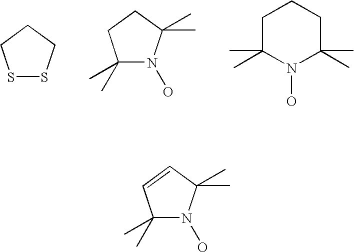
R⁴ may be lower alkyl or H; R⁵ may be selected from H, lower alkyl, -alkylene-Y and Y, wherein Y is a radical selected from the group consisting of: 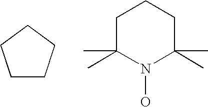
or R⁴ and R⁵ may form a group selected from the formulae: 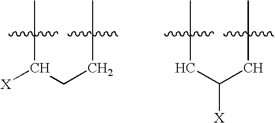
wherein X is selected from H, OH, SH, NH₂, ONO₂, SNO and NONOate; and R⁶ may be lower alkyl.

In still another preferred embodiment of this invention, a multifunctional ACE inhibitor has Formula III 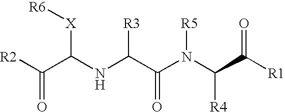
where R¹ may be selected from OH, NH₂, alkoxy, and alkyl; R² may be selected from OH, NH₂, alkoxy, and alkyl; R³ is lower alkyl; and R⁶ may be selected from -alkylene-Y and Y, wherein Y is a radical selected from the group consisting of: 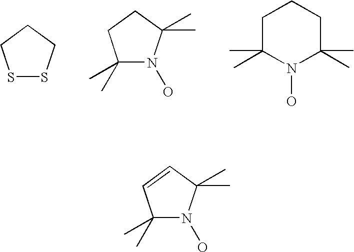
X is (CH₂)ₙ; where n an integer from 0 to 5; R⁴ is lower alkyl or H; R⁵ may be selected from H, lower alkyl, -alkylene-Y, and Y, wherein Y is a radical selected from the group consisting of: 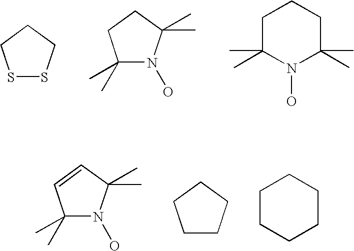
or R⁴ and R⁵ form a group independently selected from the formulae: 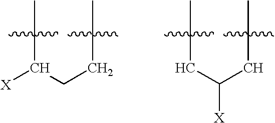
wherein X is selected from H, OH, SH, NH₂, ONO₂, SNO, and NONOate.

In a further preferred embodiment of this invention, a multifunctional ACE inhibitor has Formula IV 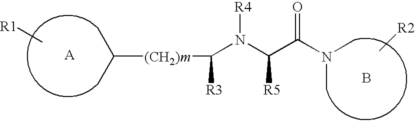
wherein m is an integer from 0 to 5; A and B are independently an optionally substituted saturated or unsaturated rings of from 4 to 18 atoms, wherein one or both comprise a ROS scavenger component; and wherein R¹ and R⁵ are independently selected from H, optionally substituted lower alkyl, and (CH₂)ₙX, where n is 0-2 and X is selected from OH, NH₂, SH, ONO, ONO₂, SNO and NONOate; R² and R³ are independently selected from COR⁶ and (CH₂)ₙX in which R⁶ is selected from OH, optionally substituted alkyl, optionally substituted acyl, optionally substituted aryl, optionally substituted heterocyclyl, and optionally substituted cycloalkyl, n is 0-2, and X is selected from OH, NH₂, SH, ONO, ONO₂, SNO, and NONOate; and R⁴ is H or lower alkyl.
Said ring A is preferably selected from the following structures 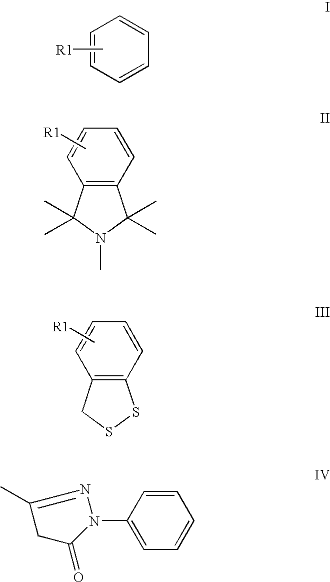
and said ring B is preferably selected from the following structures

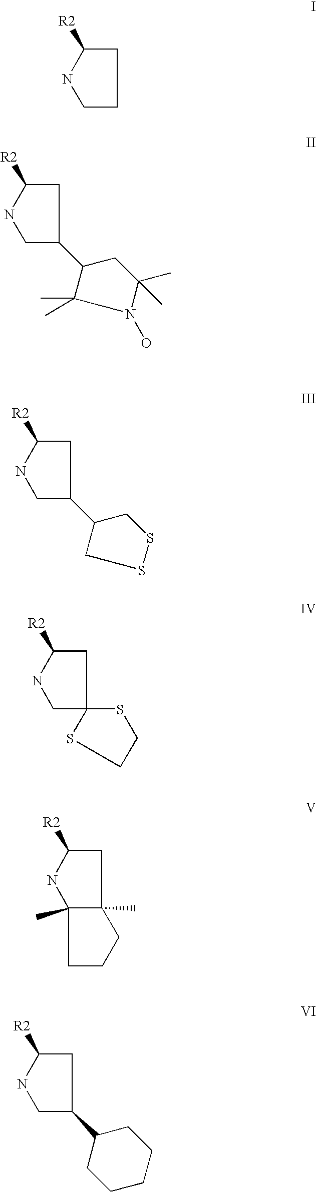

A pharmaceutical composition is further provided, comprising at least one multifunctional ACE inhibitor compound, or a solvate, optical isomer, and salt thereof, and at least one pharmaceutically acceptable excipient, diluent, propellant, etc.

### BRIEF DESCRIPTION OF THE DRAWINGS

FIG. 1 shows a model for the structure of ACE binding site.

FIG. 2 shows an active pharmacophore of ACE highlighting the binding of Captopril or Enalapril.

FIGS. 3 and 4 show the influence of a multifunctional ACE inhibitor on the blood pressure in rats.

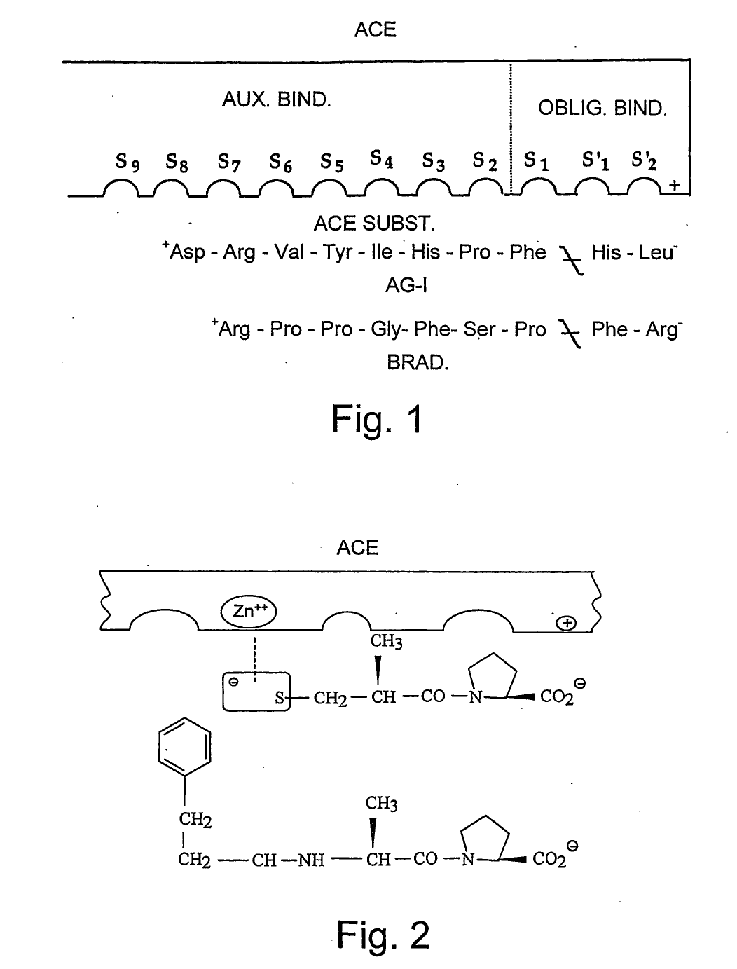

### DETAILED DESCRIPTION OF THE INVENTION

Provided are multifunctional ACE inhibitor compounds and compositions comprising said multifunctional ACE inhibitor compounds for the treatment of conditions in which treatment with ACE inhibitor compounds is indicated, and several other disorders. Cardiovascular conditions (e.g. angina, hypertension, arrhythmias, congestive heart failure) as well as other conditions (e.g. nephropathy) are the therapeutical target. The multifunctional ACE inhibitor compounds described herein are characterized in comprising at least one reactive oxygen species (ROS) scavenger component (e.g., superoxide dismutase (SOD) mimic), an ACE inhibitor component and, optionally, at least one NO donor component. The compounds may include at least one NO donor component and at least one ROS scavenger component linked to an ACE inhibitor component. In another embodiment, multifunctional ACE inhibitor compounds are provided that include at least one ROS scavenger component linked to an ACE inhibitor component, which can be made and used as described herein for multifunctional ACE inhibitors compounds.

In one embodiment of the compounds and methods described herein, the ACE inhibitor component of the multifunctional ACE inhibitor compounds described herein may comprise a derivative of, for example, the following ACE inhibitors: Captopril, Enalapril, Lisinopril, Benazepril, Fosinopril, Quinapril, Ramipril, Spirapril. Preferably, the ACE inhibitor component is selected from Captopril and Lisinopril.

In another aspect compositions are provided, including pharmaceutical compositions comprising a multifuctional ACEI compound, or its pharmaceutically acceptable salt, or its solvate, or its optical isomer, as described herein, and at least one pharmaceutically acceptable excipient, diluent, propellant, etc.

The present invention further provides use of the multifunctional ACEI compounds, and functionalized ACEI compounds described herein, as pharmaceuticals and in the manufacture of a medicament for the treatment of cardiovascular conditions involving ischemia, angina, hypertension, palpitations, arrhythmias (e.g., supraventricular, ventricular), cardiomyopathy, congestive heart failure.

The multifunctional ACE inhibitors compounds may also be employed in the treatment of conditions associated with endothelial dysfunction or oxidative stress including cardiovascular diseases (such as ischaemic heart disease, angina pectoris, myocardial infarction, congestive heart failure, atherosclerosis), diabetes mellitus, including the complications thereof (such as hypercholestemia, hypertension, atherosclerosis or Reaven's Syndrome, otherwise known as Syndrome-X), endothelial dysfunction-induced diseases, insulin-resistance and glucose intolerance in diabetes, ischemia-reperfusion tissue injury, peripheral vascular disease, critical limb ischemia, arterial aneurysms, microvascular diseases, hypertension (e.g., pulmonary, systemic, ocular, obesity or pregnancy-induced), management of arrhythmia (including but not limited to supraventricular arrhythmias, atrial tachycardia) and drug or disease induced nephropathy (e.g. diabetic nephropathy).

In some of the embodiments of the methods described herein, the multifunctional ACE inhibitors compound is administered orally. In certain embodiments of the methods as described herein, the multifunctional ACE inhibitor compound is administered via injection (e.g., intravenously, etc.). Also included in the scope of the invention are compositions of the multifunctional ACE inhibitors compounds as described herein which are formulated for delivery via injection or orally etc.

Another embodiment includes a method of treating a condition in an individual in need thereof comprising administering an effective amount of a multifunctional ACE inhibitor compound to said individual, wherein the condition is selected from the group consisting of cardiovascular conditions involving ischemia, angina, hypertension, palpitations, arrhythmias (e.g., supraventricular, ventricular), cardiomyopathy, congestive heart failure, as well as other conditions for which the use ACE inhibitors agents have proven beneficial (e.g., symptoms associated with hyperthyroidism, diabetic nephropathy, anxiety, migraine, alcohol withdrawal, tachycardia (e.g., as with thyrotoxicosis, pheochromaocytoma, reflex tachycardia), esophageal varices, wherein said ACE inhibitor compound may be selected from the compounds having Formulae A, I, II, III, or IV.

In certain methods described here, the compound is administered as a pharmaceutical composition.

In certain embodiments of the methods described herein, the condition being treated is a cardiovascular condition.

In certain embodiments of the methods described herein, the condition being treated is hypertension.

In certain embodiments of the methods described herein, the condition being treated is symptoms associated with hyperthyroidism.

In certain of the methods described herein, the multifunctional ACE inhibitor compound is administered once or twice daily.

In one aspect is provided for the treatment of the cardiovascular and other conditions for which treatment with ACE inhibitors is indicated, comprising a dosage amount of a multifunctional ACE inhibitor compound as described herein (e.g., of Formulae A, I, II, II, IV) or a composition comprising a multifunctional ACE inhibitor compound as described herein, appropriate packaging and, optionally, a delivery vehicle (e.g., pressure pack for tablets, tube for ointment, syringe for injection formulation, etc.).

The multifunctional ACE inhibitors compounds, compositions comprising the multifunctional ACE inhibitor compounds and methods for use of such multifunctional ACE inhibitor compounds described herein are also directed to avoiding adverse effects of drugs, development of tolerance (e.g., desensitization) to drugs or hypersensitivity toward drugs on repeated administration.

The multifunctional ACE inhibitor compound includes an ACE inhibitor component, a ROS scavenger component (e.g., superoxide dismutase (SOD) mimic) and, optionally, a nitric oxide donor component. Thus, in one embodiment, a known ACE inhibitor is provided in modified form and includes a superoxide dismutase (SOD) mimic component and a nitric oxide donor component capable of releasing NO in a charged or neutral form. The ACE inhibitor component may be linked to at least one ROS scavenger component and, optionally, at least one nitric oxide donor component. The superior beneficial therapeutic effects of multifunctional ACE inhibitor compounds may be attributed to their simultaneous multi-mechanistic actions as ACE inhibitor (see diverse pharmacological actions described herein), SOD-mimics and/or ROS scavengers (antioxidant and anti-inflammatory that provide additional cellular protection), and, optionally, as NO-donors (vasodilator, antioxidant, anti-proliferative, cellular protectant) at the site of drug action or therapeutic need. These properties are vital for adequate prevention and/or treatment of cardiovascular conditions involving ischemia, angina, hypertension, palpitations, arrhythmias (e.g., supraventricular, ventricular), cardiomyopathy, and congestive heart failure, as well as other conditions such as diabetic nephropathy for which the use ACE inhibitor agents are currently indicated. In another embodiment, new ACE-inhibiting structures are provided by this invention.

The multifunctional ACE inhibitor compounds and functionalized ACE inhibitor compounds described herein may also be used as pharmaceuticals or in the manufacture of a medicament for use in the treatment of conditions where treatment with an ACE inhibitor is indicated, as described herein.

In particular, described herein are nitrosated or nitrosylated ACE inhibitor agents possessing SOD-mimic and/or ROS scavenger components, which ACE inhibitors are optionally substituted with at least one ONO, SNO, or ONO₂ moiety, or a compound that donates, transfers, or releases nitric oxide in either a neutral or a charged form.

The multifunctional ACE inhibitor compounds offer a new strategy for the treatment of various diseases that can alter not only the clinical symptoms of the disease, but also its pathogenesis, natural course and outcome.

The multifunctional ACE inhibitor compounds and their compositions described herein not only provide a source of nitric oxide, which acts in the regulation of cardiopulmonary function, but also offer a direct benefit when removing injurious superoxide anion, and indirect benefit when providing ambient and endogenous protection. These properties of the multifunctional ACE inhibitor compounds make them superior over non-functionalized ACE inhibitor (e.g., higher vasodilator potency, ability to administer lower dosages, reduced toxicity). These factors prevent the development of tolerance, and reduce the toxicity levels compared to non-functionalized ACE inhibitors compounds or ACE inhibitors with NO donor alone. Additionally, oxidative stress plays an important role in the pathogenesis, progression and severity of the diseases mentioned above (cardiovascular, ocular etc) by acting in concert with other pathogenic mediators. Therefore, multifunctional ACE inhibitor compounds, have the advantage of beneficially modulating multiple pathways that determine the pathogenesis, progression and severity of the disease at the site of required drug action.

When describing the multifunctional ACE inhibitor compounds comprising one or more NO-donor component and one or more ROS scavenger component, pharmaceutical compositions comprising the multifunctional ACE inhibitor compounds and methods making or using the multifunctional ACE inhibitor compounds, the following terms have the following meanings unless otherwise specified.

As used herein, the term “multifunctional ACE inhibitor compound” refers to a compound containing an ACE inhibitor component, and additionally at least one antioxidant component, such as an ROS scavenger component, and optionally at least one NO donor component. The components may be linked, for example directly, indirectly and/or via a sharing of atoms, as described herein. The use of the term “multifunctional ACE inhibitor compound” is not intended to necessarily require that the compound was formed by chemical modification of an ACE inhibitor, since the synthesis would not necessarily involve a starting material that was an ACE inhibitor that is further modified, and other routes of synthesis are contemplated. Rather, a “multifunctional ACE inhibitor compound” is meant to be a molecule that not only includes an ACE inhibitor component with ACE inhibitor activity, but also the additional functionality of the antioxidant (such as ROS scavenger) and NO donor. Thus, in one embodiment, multifunctional ACE inhibitor compounds are provided that are ACE inhibitor in a modified form wherein they include an NO donor component and a ROS scavenger component.

NO Donors

Groups that can act as nitric oxide donors are capable of acting as a source of nitric oxide (NO). The nitric oxide donor component is, for example, an —ONO₂—ONO, —SNO or —(NO)₂ group. In particular embodiments the NO donor component is —ONO₂ or —SNO. The NO donor component, for example, donates, transfers, or releases nitric oxide in either a neutral or a charged form. The nitric oxide donor component may comprise any group capable of acting as a source of nitric oxide (NO) in a charged or uncharged form, including nitrosonium (NO+), nitroxyl (NO−) or nitric oxide (NO.).

Reactive Oxygen Species Scavengers

The multifunctional ACE inhibitor compound may include a chemical moiety that can function as an antioxidant component, preferably without affected the stability and action of the NO donor component, as well as the NO donor component. The antioxidant component can be a reactive oxygen species (ROS) scavenger. As used herein, the term “reactive oxygen species (ROS) scavenger component” refers to a moiety capable of acting as a scavenger of, or reacting with, superoxide (O₂⁻) or other reactive oxygen species (ROS) including hydroxyl radicals, peroxynitrite, hypochlorous acid and hydrogen peroxide. An antioxidant that preferentially scavenges, or reacts with, superoxide is termed a “superoxide dismutase mimic” (SOD-mimic), superoxide scavenger, or “superoxide dismutase mimetic” (SOD-mimetic). The reactive oxygen species superoxide (O₂⁻), hydroxyl radicals, peroxynitrite, hypochlorous acid and hydrogen peroxide are considered biologically undesirable, while nitric oxide, as described above, may be biologically beneficial. Thus, the antioxidant or ROS scavenger component preferably does not react with, or scavenge, nitric oxide.

The multifunctional ACE inhibitor compounds described herein may include one or more antioxidant or ROS scavenger components. In some embodiments, the reactive oxygen species scavenger component is a nitroxide free radical (NO.) group. In certain embodiments the compounds as described herein may comprise more than one ROS scavenger component, for example at least one, at least two, at least three or at least four ROS scavenger components.

As used herein, the ROS scavenger component itself is not intended to be a group capable of donating nitric oxide (NO). Further, the ROS scavenger component is provided in addition to the ACE inhibitor component of the multifunctional ACE inhibitor compound.

The antioxidant component, such as an ROS scavenger component, may be for example an alkenyl group; aryl group; substituted aryl group, where the aryl group is substituted with, for example, —OH, —NH₂, —NHCHO or a NO donor group; sulfhydryl (in a protected form) or dithiol in oxidized or reduced form; or a group that is, or is capable of being converted in vivo into, a sulfhydryl in its oxidized or reduced form.

In particular embodiments, the ROS scavenger component may be an N-oxide free radical, wherein optionally the nitrogen of the N-oxide free radical is within a 3-, 4-, 5-, 6- or 7-membered ring, wherein the ring may be substituted or unsubstituted with, for example, straight or branched chain C₄-C₇, or C₁-C₃ alkyl groups, alkoxy groups and groups capable of donating NO.

The N-oxide free radical is preferably substituted. In particular embodiments the N-oxide free radical is fully substituted at positions alpha to the nitroxide free radical, and may optionally be substituted at other positions on the ring. Exemplary substituents for the alpha positions include methyl or ethyl. Exemplary substituents for other ring positions include NO donor groups.

The nitrogen of the substituted N-oxide free radical may also be linked to the ACE inhibitor at the backbone amine of the ACE inhibitor.

In certain other embodiments the substituted N-oxide free radical may also be substituted within the ring with an additional heteroatom, for example, —O— or —S—, (see structures Ia and Ib, below). Exemplary substituted N-oxide free radicals include substituted pyrrolidinyloxy free radicals (e.g., PROXYL), substituted piperidinyloxy free radicals (e.g., TEMPO), substituted oxazolidinyloxy free radicals (e.g., DOXYL), substituted oxazinyloxy free radicals, substituted thiazolidinyloxy free radicals and substituted thiazinyloxy free radicals.

In certain embodiments, the ROS scavenger(s) may be independently selected from the group consisting of substituted piperidinyloxy free radical, substituted 3-pyrrolidin-1-yloxy free radical, substituted oxazolidinyloxy free radical (e.g., DOXYL), and an substituted or unsubstituted lipoic acid moiety.

Examples of substituted N-oxide free radical moieties which may be incorporated into the multifunctional ACE inhibitor compounds include a 2,2,6,6-tetramethylpiperidinyloxy free radical (TEMPO) moiety (Ia, below; where X═C), a 2,2,5,5-tetramethyl-3-pyrrolidin-1-yloxy free radical (PROXYL) moiety (Ib, below, where X═C); 4,4-dimethyl-3-oxazolidinyloxy (DOXYL) free radical moiety, and a 2,2,4,4-tetramethyl-3-oxazolidinyloxy free radical moiety (Ib, below, where X═O). In structures Ia-f below, X is for example —S—, —C— or —O—. The substituted N-oxide free radical moiety may be linked to the ACE inhibitor moiety for example, directly, indirectly, via a linker (e.g., through an alkyl substituent group, see, for example Ic and Id), and/or via sharing of atoms, for example as shown in structures Ie and If below. The linkage may be to various carbon atoms on the ring, including those shown in structures Ic-If below. Additionally, the substituted N-oxide free radical moiety may be linked to the ACE inhibitor component via incorporation in a fused ring system.

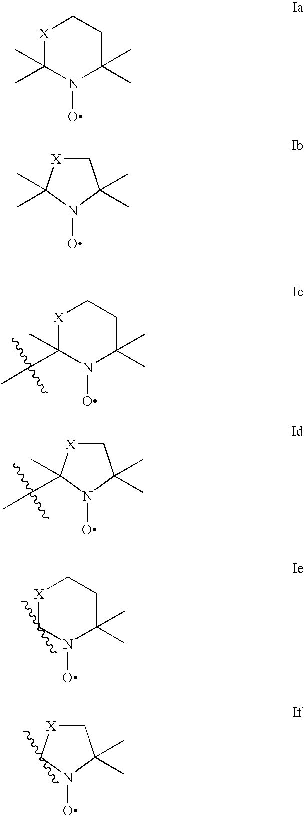

In other embodiments the ROS scavenger component comprises a lipoic acid moiety or may be derived from the lipoic acid moiety. The lipoic acid moiety may be substituted or unsubstituted and is shown below:

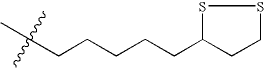

The lipoic acid moiety may be independently substituted by one or more groups such as straight or eventually branched chain C₁-C₁₅ alkyl groups, C₁-C₁₅ alkoxy groups, hydroxy groups, amino groups, —NHCHO groups, —CH₂OH groups, and groups capable of donating NO in a charged or neutral form.

In other embodiments, the ROS scavenger component may be a pantothenic acid SH-containing derived moiety as shown below, in either an oxidized or reduced form: 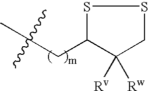
wherein, m is for example, 1-6, and R^{v} and R^{w} are for example independently C₁-C₃ alkyl or H.

In other embodiments, the lipoic acid moiety may be modified by varying the length of the aliphatic chain connecting the heterocyclic ring to the ACE inhibitor component of the multifunctional ACE inhibitor compound. The chain may be for example (CH₂)ₙ wherein n is an integer from 1-15. In certain embodiments n is 2-12, in particular embodiments, n is 3 or 12 as shown below.

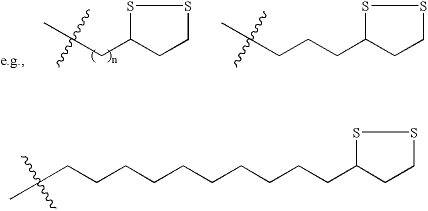

The ROS scavenger/SOD mimic component may also comprise a substituted N-oxide free radical, where the nitrogen of the N-oxide free radical is contained with a cyclic ring (e.g., a 5-, 6-, or 7-membered ring) and is linked to the ACE inhibitor at the backbone amine of the ACE inhibitor component. Exemplary N-oxide free radicals are shown below, where the NH as pictured below may form part of the ACE inhibitor component.

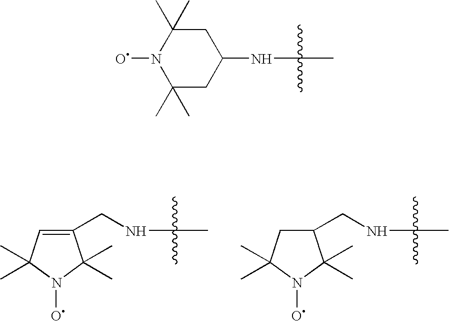

In some embodiments, the ROS scavenger/SOD mimic component may comprise a substituted or unsubstituted S—S-containing ring (e.g., 5-, 6-, or 7-membered ring), e.g., as shown below, where the NH as pictured below may form part of the ACE inhibitor component.

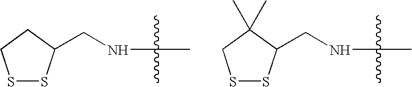

In certain embodiments the ROS scavenger component, including those described above, may be independently substituted with one or more alkyl groups such as C₁-C₁₅ alkyl groups, alkoxy such as C₁-C₁₅ alkoxy groups, hydroxy groups, amino groups, —NHCHO groups, —CH₂OH groups, and groups capable of donating NO in a charged or neutral form.

In particular embodiments, the ROS scavenger component (s) comprises, one or more PROXYL moieties, one or more TEMPO moieties, one or more DOXYL moieties, one or more 2,2,4,4-tetramethyl-3-oxazolidinyloxy free radical moieties and/or one or more substituted or unsubstituted lipoic acid moieties. In particular embodiments the groups comprising N-oxide free radical moieties are independently substituted by one or more C₁-C₄ alkyl groups, for example methyl, ethyl or butyl, or one or more C₁-C₄ alkoxy groups.

The multifunctional ACE inhibitor compounds may be modified to include one or more of the same or different SOD mimic component and/or ROS scavenger component.

ACE Inhibitors

The ACE inhibitor component of any of a variety of ACE inhibitor compounds for the treatment of cardiovascular and other conditions disclosed herein can be present in the multifunctional ACE inhibitor compounds. In one embodiment, a known ACE inhibitor is provided in a derivatized, multifunctional form that further includes at least one NO donor component and at least one ROS scavenger component. The ACE inhibitor compound or component has an affinity for ACE molecule; the ACE inhibitor compound or component is one that is capable of inhibiting angiotensin converting enzyme. After incorporating one of the two activities, namely inhibiting ACE and scavenging ROS, into a molecule that has only one of them, a multifunctional ACE inhibitor of this invention is obtained. After incorporating one of the three activities, namely inhibiting ACE or scavenging ROS or donating NO, into a compound that has only two of them, a preferred multifunctional ACE inhibitor compound according to this invention is obtained. The multifunctional ACE inhibitor compounds may be used to treat any of the indications for which treatment with ACE inhibitor is indicated.

Exemplary ACE inhibitors include compounds used in the treatment of cardiovascular conditions and others described herein that selectively inhibit ACE. ACE inhibitor agents remain the cornerstone for therapy of all stages of ischemic heart disease. They constitute the standard therapy for effort hypertension, angina, mixed effort and rest angina, and unstable angina. They decrease mortality in acute-phase myocardial infarction and in the post-infarct period. In addition to their primary role in the treatment of ischemic heart disease, ACE inhibitors retain their leading position among basic therapies for other cardiovascular conditions including hypertension, arrhythmias, cardiomyopathy, and congestive heart failure. ACE inhibitors also possess other properties that make them useful for the treatment of non-cardiovascular conditions such as diabetic nephropathy. ACE inhibitors are now recognized as an integral part of antihypertension therapy. However, despite the increasingly impressive results of ACE inhibitor therapy, the mechanisms of action are still unclear. The multifunctional compounds of this invention provide new possibilities in the mentioned treatments; and new mechanisms will probably be involved as well.

Substituents

As used herein, the term “alkyl” includes branched or unbranched hydrocarbon chains, for example, including about 1 to about 18 carbons, or 1-5 carbons, such as methyl, ethyl, n-propyl, iso-propyl, n-butyl, sec-butyl, iso-butyl, tert-butyl, octa-decyl and 2-methylpentyl. Alkyl may also include cyclic alkyl groups, for example, including about 5-8 carbons, such as cyclopentyl, cyclohexyl, cycloheptyl, or cycloctyl. The term “lower alkyl” refers to an alkyl group having from 1 to 6 carbon atoms. Alkyl can be substituted or unsubstituted with one or more functional groups such as hydroxyl, bromo, fluoro, chloro, iodo, mercapto or thio, cyano, alkylthio, aryl, carboxyl, carbalkoyl, alkenyl, nitro, amino, alkoxyl, amido, an NO donor group, and the like in the form of substituted alkyl. A cyclic alkyl group may be substituted with a straight or branched chain alkyl group.

Substituted alkyl groups may also refer to an alkyl group having from 1 to 5 substituents, or from 1 to 3 substituents, such as, acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— or aryl-S(O)₂—.

As used herein, reference to alkylene groups means a branched or unbranched, cyclic or acyclic, saturated or unsaturated (e.g. alkenylene or alkynylene) hydrocarbylene radical. Where cyclic, the alkylene group is preferably C₃ to C₁₂, more preferably C₅ to C₇. Where acyclic, the alkylene group is preferably C₁ to C₁₆, more preferably C₁ to C₄, still more preferably methylene.

The term “heteroaryl” includes a ring system including one or more aromatic rings and containing one or more heteroatoms, N, O, or S, in the aromatic ring. Heteroaryl groups can be unsubstituted or may be substituted for example as described for alkyl and aryl groups. Examples of heteroaryl groups include, but are not limited to, pyridinyl, pyrazinyl, pyrimidinyl, benzothialozyl, pyrazolyl, benzoxazolyl, imidazolyl, pyrrolyl, thiadiazolyl, oxazolyl, isoxazolyl, pyridazinyl, triazolyl, thiazolyl, isothiazolyl, thiophenyl, furanyl, and quinolinyl.

The term “alkoxy” includes the group —OR^{d} where R^{d} is substituted or unsubstituted alkyl (e.g., 1-10 carbons, or 1-4 carbons). In some embodiments, the alkoxy groups may be, for example, independently, methoxy, ethoxy, n-propoxy, isopropoxy, n-butoxy, tert-butoxy, sec-butoxy, n-pentoxy, or n-hexoxy, 1,2-dimethylbutoxy.

The term “substituted alkoxy” includes an alkoxy group having from 1 to 5 substituents (e.g., 1-5 or 1 to 3 substituents), where the substituents may independently include substituted or unsubstituted acyl, substituted or unsubstituted acylamino, substituted or unsubstituted acyloxy, substituted or unsubstituted alkoxy, alkoxycarbonyl, alkoxycarbonylamino, substituted or unsubstituted amino, aminocarbonyl, aminocarbonylamino, aminocarbonyloxy, substituted or unsubstituted aryl, aryloxy, azido, carboxyl, cyano, substituted or unsubstituted cycloalkyl, halogen, hydroxyl, keto, nitro, substituted or unsubstituted thioalkoxy, substituted or unsubstituted thioaryloxy, thioketo, thiol, alkyl-S(O)—, aryl-S(O)—, alkyl-S(O)₂— or aryl-S(O)₂—.

The term “alkoxycarbonyl” includes the group —C(O)OR^{e} where R^{e} may be substituted or unsubstituted alkyl optionally or substituted cycloalkyl.

“Alkoxycarbonylamino” includes the group —NR^{f}C(O)OR^{g}, where R^{f} may be, for example, hydrogen, substituted or unsubstituted alkyl, substituted or unsubstituted aryl or substituted or unsubstituted cycloalkyl, and R^{g} may be, for example, substituted or unsubstituted alkyl or substituted or unsubstituted cycloalkyl.

The term “substituted amino” includes groups such as —N(R^{h})₂ where each R^{h} may independently be, for example, hydrogen, alkyl, substituted alkyl, alkenyl, substituted alkenyl, alkynyl, substituted alkynyl, substituted or unsubstituted aryl, cycloalkyl, substituted cycloalkyl, or where the R^{h} groups join to form an substituted or unsubstituted alkylene group. When both R^{h} groups are hydrogen, —N(R^{h})₂ is an amino group.

The term “aminocarbonyl” includes groups such as —C(O)NR^{j}R^{k} where R^{j} and R^{k} may independently be hydrogen, alkyl, aryl and cycloalkyl, or where R^{j} and R^{k} join to form an alkylene group, which is substituted or unsubstituted.

The term “aminocarbonylamino” includes groups —NR¹C(O)NR^{m}Rⁿ where R¹, R^{m}, and Rⁿ may independently be hydrogen, alkyl, aryl and cycloalkyl, or where R^{m} and Rⁿ join to form an alkylene group, which is substituted or unsubstituted.

The term “aminocarbonyloxy” includes groups such as —OC(O)NR^{p}R^{q} where R^{p} and R^{q} may independently be hydrogen, alkyl, aryl and cycloalkyl, or where R^{p} and R^{q} join to form an alkylene group, which is substituted or unsubstituted.

The term “cycloalkyl” includes cyclic alkyl groups of, for example, 3 to 10 carbon atoms having a single cyclic ring or multiple condensed or bridged ring. The rings may be substituted or unsubstituted with from, for example, 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, or multiple or bridged ring structures such as adamantanyl and the like. The term “lower cycloalkyl” refers to a cycloalkyl group having from 3 to 6 carbon atoms.

“Substituted cycloalkyl” includes cycloalkyl groups having, for example, from 1 to 5 substituents, or from 1 to 3 substituents, where the substituents may independently include, for example, substituted or unsubstituted acyl, acylamino, acyloxy, substituted or unsubstituted alkoxy, alkoxycarbonyl, alkoxycarbonylamino, substituted or unsubstituted amino, aminocarbonyl, aminocarbonylamino, aminocarbonyloxy, substituted or unsubstituted aryl, aryloxy, azido, carboxyl, cyano, substituted or unsubstituted cycloalkyl, halogen, hydroxyl, keto, nitro, substituted or unsubstituted thioalkoxy, substituted or unsubstituted thioaryloxy, thioketo, thiol, alkyl-S(O)—, aryl-S(O)—, alkyl-S(O)₂— or aryl-S(O)₂—.

“Cycloalkoxy” includes groups —OR^{t} where R^{t} may be, for example, cycloalkyl, as described above. Such cycloalkoxy groups include, by way of example, cyclopentoxy, cyclohexoxy and the like.

Multifunctional ACE Inhibitor Compounds

The multifunctional ACE inhibitor compound includes an ACE inhibitor component, at least one antioxidant component such as a reactive oxygen species (ROS) scavenger (e.g., a SOD mimic), and optionally at least one NO-donor component. The multifunctional ACE inhibitor compound may include an ACE inhibitor component linked to at least one NO-donor component and at least one antioxidant component. The term “linked” as used herein is intended to include direct and indirect linkages and shared atoms (including, for example, where the nitrogen of the substituted N-oxide free radical is part of a fused ring system) between any of the NO donor component, antioxidant component, such as ROS scavenger component, and ACE inhibitor component. The components may be linked in any order, for example, the ROS scavenger component may be linked to a molecule that comprises both the NO donor component and the ACE inhibitor component, or the ROS scavenger component may be linked only to the ACE inhibitor component, etc., attaining structures, e.g., according to Formulae I-IV.

In some embodiments, functionalized ACE inhibitor compounds are provided that include at least one ROS scavenger component (e.g., SOD mimic) linked to an ACE inhibitor component, which can be made and used as described herein for multifunctional ACE inhibitor compounds.

Also included within the scope of the invention are salts of the compounds disclosed herein and stereoisomers thereof. The compounds of the present invention contain one or more asymmetric atoms and may exist in diastereomeric, racemic and optically active forms. All such compounds and compositions comprising these compounds are within the scope of this invention. Therefore, where a compound is chiral, the separate enantiomers, substantially free of the other, are included within the scope of the invention. Thus, one enantiomer may be in, for example, 95% or more purity. Further included are all mixtures of enantiomers or diastereomers.

Optically active forms of the compounds can be prepared using any method known in the art, including by resolution of the racemic form by recrystallization techniques, by chiral synthesis, extraction with chiral solvents, or by chromatographic separation using a chiral stationary phase. Examples of methods to obtain optically active materials include transport across chiral membranes, a technique whereby a racemate is placed in contact with a thin membrane barrier. The concentration or pressure differential causes preferential transport across the membrane barrier. Separation occurs as a result of the non-racemic chiral nature of the membrane which allows only one enantiomer of the racemate to pass through. Chiral chromatography, including simulated moving bed chromatography, is used in one embodiment. A wide variety of chiral stationary phases are commercially available.

Since superoxide anion is an available and continuously-formed by-product generated through normal metabolic processes, and since its elimination is mediated either by dismutation by the enzyme SOD or via its reaction with NO to form the potentially hazardous peroxynitrite, without being limited to any theory, the compounds are believed to be capable of simultaneously and favorably affecting both components; the NO and O₂⁻. By virtue of the ACE inhibitor activity, NO donation and superoxide scavenging properties being simultaneously delivered by the same molecule, the compounds of the present invention can increase the level of NO and reduce levels of superoxide thereby avoiding high levels of peroxynitrite and oxidant metabolites thereof and consequently increasing the effectiveness of the ACE inhibitor component (as removal of superoxide anions leads to lower responses to angiotensin since superoxide anions (generated by NADPH oxidase) partly mediates the biological responses to angiotensin). Furthermore, the multifunctional ACE inhibitor compounds as described herein can also be administered at more predictable doses compared to non-functionalized ACE inhibitor due to the ability of the ROS scavenger component to scavenge ROS species which can interfere with ACE inhibitor activity or NO donated in vivo.

Therefore one embodiment of the invention provides multifunctional ACE inhibitor compounds comprising a functionalized ACE inhibitor component which contains at least one moiety that affords SOD-mimic and/or ROS scavenger activity, and at least one ONO, SNO, or ONO₂ component that confers on the ROS scavenger-ACE inhibitor an additional relaxant effect with all other beneficial biological actions expected from an NO-donor. In other embodiments, functionalized ACE inhibitor compounds are provided that include at least one ROS scavenger and/or SOD mimic component linked to an ACE inhibitor component, which can be made and used as described herein for multifunctional ACE inhibitor compounds.

In some embodiments the at least one ROS scavenger component may be a SOD mimic.

The nitric oxide donor components may include ONO, —ONO₂, —SNO and —(NO)₂.

The antioxidant component, such as a ROS scavenger component is, for example, a substituted N-oxide free radical, wherein the nitrogen of the N-oxide is contained within a ring (e.g., a 5-, 6-, or 7-membered ring); alkenyl group; aryl group; substituted aryl group, where the aryl group is substituted with, for example, —OH, —NH₂, —NHCHO or a NO donor group; or a group that is, or is capable of being converted in vivo into, a sulfhydryl in oxidized or reduced form (e.g., a group incorporating a lipoic acid moiety).

In some embodiments, novel multifunctional ACE inhibitor compounds are provided comprising an ACE inhibitor component, at least one NO-donor component and at least one superoxide anion (O₂⁻) scavenger component and their use as therapeutic agents for the treatment of cardiovascular conditions and other conditions in which treatment with ACE inhibitors is indicated without producing undesired side effects

Consequently, the present invention relates to ACE inhibitor agents with either SOD or anti-ROS activity, optionally possessing NO donation properties of the general Formulae I, II, III, and IV. The anticipated superior beneficial therapeutic effects of compounds comprising these Formulae may be attributed to their simultaneous multi-mechanistic actions as ACE inhibitor (see diverse pharmacological actions above), SOD-mimics/anti-ROS (antioxidant and anti-inflammatory that provide additional cellular protection), and as NO-donors (vasodilator, antioxidant, anti-proliferative, cellular protectant with potent vascular smooth muscle relaxing properties). These properties are most needed for adequate prevention and/or treatment of cardiovascular conditions involving ischemia, hypertension, arrhythmias, cardiomyopathy, congestive heart failure, as well as other conditions for which the use of ACE inhibitor components has proven beneficial (diabetic nephropathy).

In particular, the invention relates to nitrosated or nitrosylated ACE inhibitor agents with SOD-mimic/Anti-ROS actions which can optionally be substituted with at least one ONO, SNO, or ONO₂ moiety, or a compound that donates, transfers, or releases nitric oxide in either a neutral or a charged form.

The suggested compounds offer a new strategy for the treatment of various diseases that can affect not only the clinical symptoms of the disease, but also its pathogenesis, natural course and outcome.

In one embodiment, the ROS scavenger is a SOD mimic (e.g., substituted pyrolidinyloxy N-oxide free radical); at least one NO donor group is ONO, SNO or ONO₂; and the ACE inhibitor component can be either Captopril or Lisinopril.

In one embodiment of this invention, a multifunctional ACE inhibitor has formula A:

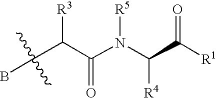

where

R¹ may be independently selected from hydrogen (H), alkyl, hydroxyl (OH), amino (NH₂), alkoxy (preferably lower alkoxy such as OCH₃, OCH₂CH₃, OCH(CH₃)₂, OC(CH3)₃);

R³ may be independently selected from lower alkyl, -alkylene-Y or Y, wherein Y is a radical selected from the group consisting of:

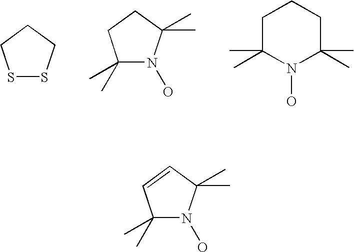

Preferably, R³ may be independently Y, wherein Y is

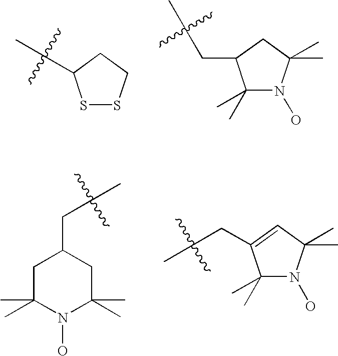

R⁴ and R⁵ may be independently selected from lower alkyl, H or together form a group selected from the formulae:

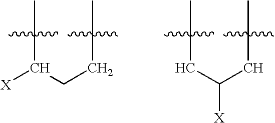

wherein X is defined as H, OH, SH, NH2, ONO₂, SNO or N(NO)₂

R⁵ may also be independently selected from -alkylene-Y or Y, wherein Y is a radical selected from the group consisting of:

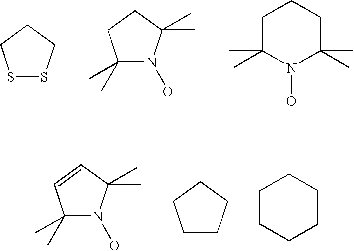

and wherein B may be independently selected from:

i) R², where R² may be independently selected from H or lower alkyl, preferably CH₃;

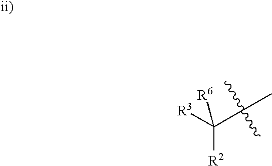

wherein: R² may be independently selected from SH or SNO,
R³ may be independently selected from -alkylene-Y or Y, wherein Y is a radical selected from the group consisting of:

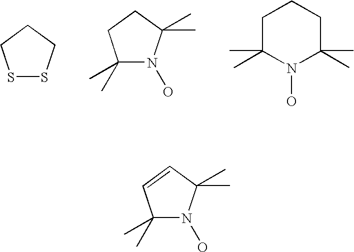

Preferably, R³ may be independently Y, wherein Y is

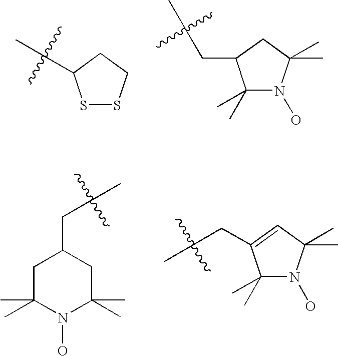

R₆ may be lower alkyl, preferably CH₃;

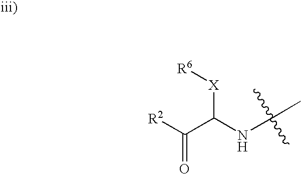

wherein R² may be independently selected from hydroxyl (OH), amino, alkoxy (preferably lower alkoxy, such as OCH₃, OCH₂CH₃, OCH(CH₃)₂ or OC(CH₃)₃), or alkyl (preferably iso-butyl, pentyl or iso-pentyl);

R⁶ may be independently selected from -alkylene-Y or Y, wherein Y is a radical selected from the group consisting of:

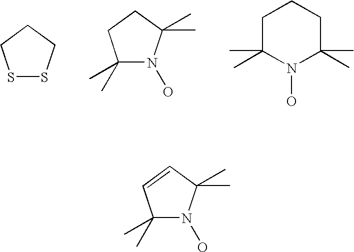

Preferably, R⁶ may be independently Y, wherein Y is 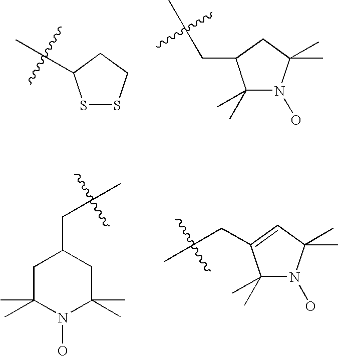
and X is defined as (CH₂)ₙ; where n is equal to 0-5

In a preferred embodiment of this invention, a multifunctional ACE inhibitor has Formula I:

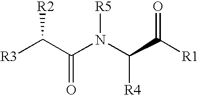

where

R¹ may be independently selected from hydrogen (H), hydroxyl (OH), amino (NH₂), alkoxy (preferably lower alkoxy, such asOCH₃, OCH₂CH₃, OCH(CH₃)₂, OC(CH3)₃);

R² may be independently selected from hydrogen (H) and lower alkyl (preferably CH₃);

R³ may be independently selected from -alkylene-Y or Y, wherein Y is a radical selected from the group consisting of:

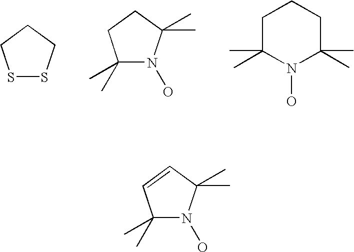

Preferably, R³ may be independently Y, wherein Y is

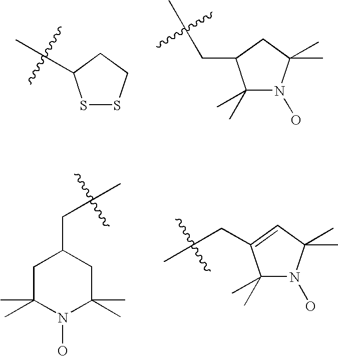

R⁴ may be lower alkyl or preferably H;

R⁵ may be independently selected from H, lower alkyl or from -alkylene-Y or Y, wherein Y is a radical selected from the group consisting of:

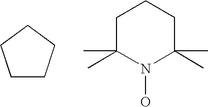

Alternatively, R⁴ and R⁵ may together form a group independently selected from the formulae:

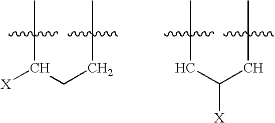

wherein X is selected from H, OH, SH, NH₂, ONO₂, SNO and NONOate.

In another preferred embodiment of this invention, a multifunctional ACE inhibitor has Formula II:

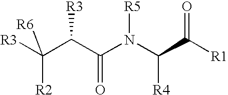

where R¹ may be independently selected from hydrogen (H), hydroxyl (OH), amino (NH₂), alkoxy (preferably lower alkoxy such as OCH₃, OCH₂CH₃, OCH(CH₃)₂, OC(CH₃)₃);

R² may be independently selected from SH, SNO;

R³ may be independently selected from -alkylene-Y or Y, wherein Y is a radical selected from the group consisting of:

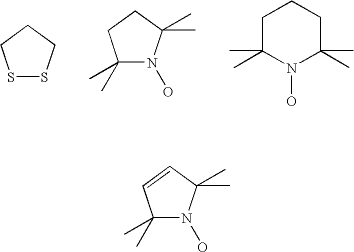

Preferably, R³ may be independently Y, wherein Y is

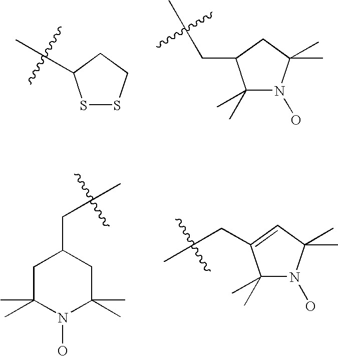

R⁴ may be lower alkyl or preferably H;

R⁵ may be independently selected from H, lower alkyl or from -alkylene-Y or Y, wherein Y is a radical selected from the group consisting of:

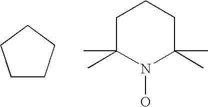

Alternatively, R⁴ and R⁵ may together form a group independently selected from the formulae:

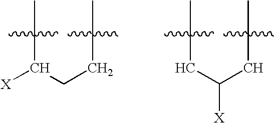

wherein X is defined as H, OH, SH, NH₂, ONO₂, SNO or N(NO)₂

R⁶ may be lower alkyl, preferably CH₃

In a further preferred embodiment of this invention, a multifunctional ACE inhibitor has Formula III:

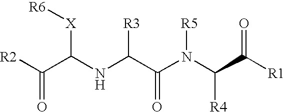

where

R¹ may be independently selected from hydroxyl (OH), amino (NH2), alkoxy (preferably lower alkoxy, such as OCH₃, OCH₂CH₃, OCH(CH₃)₂, OC(CH₃)₃) or alkyl (preferably iso-butyl, pentyl, iso-pentyl);

R² may be independently selected from hydroxyl (OH), amino, alkoxy (OR) (preferably lower alkoxy, such as OCH₃, OCH₂CH₃, OCH(CH₃)₂, OC(CH₃)₃) or alkyl (preferably iso-butyl, pentyl, iso-pentyl);

R³ may be independently selected from lower alkyl, preferably methyl

R⁶ may be independently selected from -alkylene-Y or Y, wherein Y is a radical selected from the group consisting of:

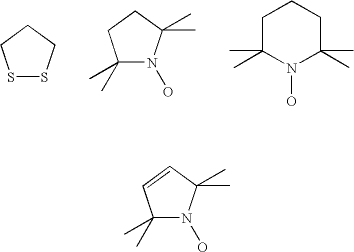

Preferably, R⁶ may be independently Y, wherein Y is

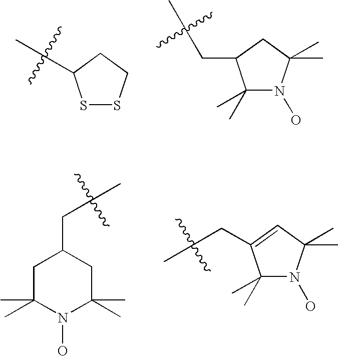

X is defined as (CH₂)ₙ; where n is equal to 0-5 R⁴ may be lower alkyl or preferably H;

R⁵ may be independently selected from H, lower alkyl or -alkylene-Y or Y, wherein Y is a radical selected from the group consisting of:

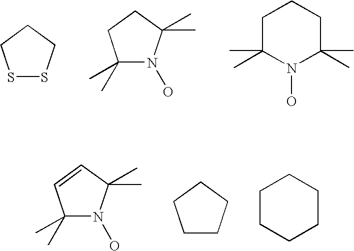

Alternatively, R⁴ and R⁵ may together form a group independently selected from the formulae:

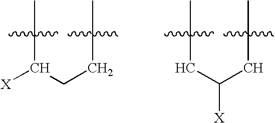

wherein X is selected from H, OH, SH, NH₂, ONO₂, SNO and NONOate.

In still another preferred embodiment of this invention, a multifunctional ACE inhibitor has Formula IV:

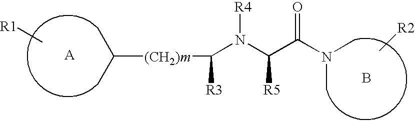

wherein:

m is an integer with a value ranging from zero to five;

A and B are independently an optionally substituted saturated or unsaturated ring of from 4 to 18 atoms, wherein either or both A and B comprise a ROS scavenger component;

R¹ is selected from H and optionally substituted lower alkyl;

R² and R³ are independently selected from formula COR⁶ or (CH₂)ₘX

wherein

R⁶ is selected from hydroxyl, optionally substituted alkyl, optionally substituted acyl, optionally substituted aryl, optionally substituted heterocyclic and optionally substituted cycloalkyl groups

n is 0-2;

X is OH, NH₂, SH, ONO, ONO₂, SNO and N(NO)₂;

R⁴ is selected from hydrogen and lower alkyl, preferably methyl;

R⁵ is selected from hydrogen and lower alkyl.

A is an optionally substituted saturated or unsaturated ring system of from 4 to 18 atoms, preferably 4 to 7 atoms. The ring system may be saturated or unsaturated, aromatic or non-aromatic, carbocyclic or heterocyclic, monocyclic or polycyclic (ie comprise two or more rings which may be fused or non-fused). Preferably A is an optionally substituted, mono- or bi-cyclic, fused or non-fused phenyl group. In one embodiment, Ring A is selected from the following ring systems:

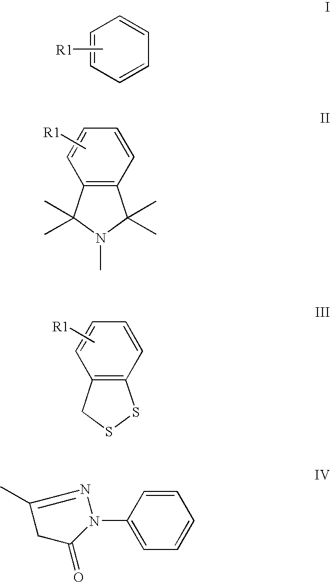

B is an optionally substituted, saturated or unsaturated ring system of from 4 to 18 atoms, preferably 4 to 7 atoms, more preferably 5 atoms, and including a nitrogen atom. The ring system may be saturated or unsaturated, aromatic or non-aromatic, monocyclic or polycyclic (ie comprise two or more rings which may be fused or non-fused). Preferably A is an optionally substituted, mono- or bi-cyclic, fused or non-fused pyrrolidinyl group. In one embodiment, Ring B is selected from the following ring systems:

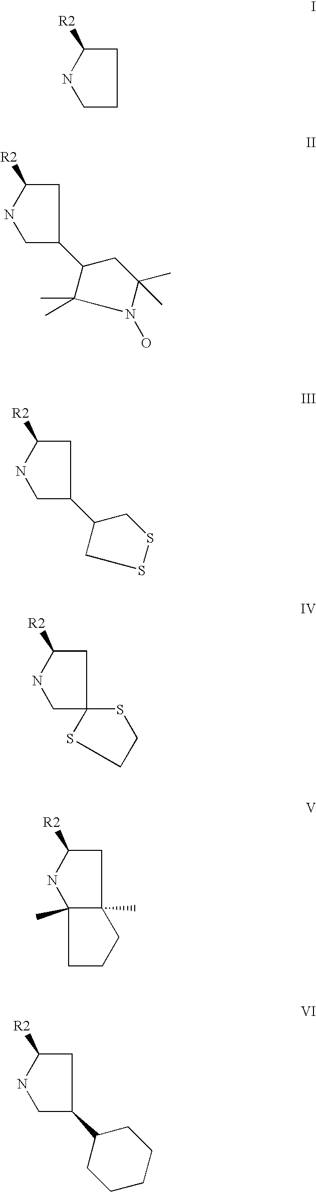

The compounds of Formulae I, II, III, and IV have preferably at least one SOD mimic component which is a substituted N-oxide free radical in which the nitrogen of the N-oxide group of the substituted N-oxide free radical is within a 5- or 6-membered ring. In a preferred embodiment, at least one substituted N-oxide free radical is independently selected from the group consisting of pyrrolidinyloxy free radicals, piperidinyloxy free radicals, oxazolidinyloxy free radicals, oxazinyloxy free radicals, thiazolidinyloxy free radicals and thiazinyloxy free radicals. In another embodiment, the substituted N-oxide free radical is a substituted 3-oxazolidinyloxy free radical. In another embodiment, the compound comprises at least two nitric oxide donor components.

In another embodiment, compounds according to Formulae I or II or III or IV are provided where the compound includes one or more ROS scavenger components but does not include an NO donor group.

The multifunctional ACE inhibitor compounds of this invention include, but are not limited to, compounds of Formulae I, II, III, IV as described herein. In one embodiment of the invention, multifunctional and functionalized ACE inhibitor compounds are provided, as well as compositions comprising them, and methods for their use in treating diseases.

In another of its composition aspects, this invention is directed to pharmaceutical compositions comprising a pharmaceutically acceptable carrier and a pharmaceutically effective amount of a multifunctional ACE inhibitor compound of the present invention, preferably of formula I or II or III or IV.

In some embodiments, the compound comprises at least two nitric oxide donor components.

Synthesis of Multifunctional ACE Inhibitor Compounds

Multifunctional ACE inhibitor compounds may be synthesized as described herein using methods available in the art and standard techniques in organic chemistry, as described, for example, in March's Advanced Organic Chemistry: Reactions, Mechanisms, and Structure, 5th Edition (2000) M. B. Smith & J. March, John Wiley & Sons, New York, N.Y.; Organic Chemistry 6^{th} Ed. (1992) R. Morrison & R. Boyd, Benjamin Cummings, San Francisco; and Richard C. Larock, “Comprehensive Organic Transformations” New York: Wiley-VCH; 1989.

The multifunctional ACE inhibitor compounds of Formulae I, II, III, and IV can be prepared from readily available starting materials using the general methods and procedures, as described in Examples. The general approach for synthesis of preferred compounds of this invention is outlined in the following Examples.

Methods of Use for Multifunctional Ace Inhibitor Compounds

The present invention provides the multifunctional ACE inhibitor compounds for use in the treatment of cardiovascular and other conditions for which treatment with ACE inhibitor is indicated, as described herein.

The present invention further provides the use of the multifunctional ACE inhibitor compounds and functionalized ACE inhibitor compounds of the present invention in the manufacture of a medicament for the treatment of cardiovascular conditions involving ischemia, angina, hypertension, palpitations, arrhythmias (e.g., supraventricular, ventricular), cardiomyopathy, congestive heart failure, as well as other conditions for which the use ACE inhibitor agents have proven beneficial (e.g., diabetic nephropathy).

The multifunctional ACE inhibitor compounds may also be employed in the treatment of conditions associated with endothelial dysfunction or oxidative stress including cardiovascular diseases (such as ischaemic heart disease, angina pectoris, myocardial infarction, congestive heart failure, atherosclerosis, hypertension (e.g., pulmonary, systemic, ocular, obesity or pregnancy-induced), and management of arrhythmia (including but not limited to supraventricular arrhythmias, atrial tachycardia).

The relationship between reactive oxygen species (ROS) and nitric oxide (NO) plays a detrimental role in the modulation of many biological processes including aging, atherosclerosis, hypertension, diabetes mellitus, degenerative conditions, carcinogenesis, ischemia-reperfusion tissue injury, and acute and chronic inflammatory conditions. This is especially true in the case of cardiovascular conditions in general, and in hypertension and in particular, as well as in other conditions as indicated above (e.g., thyrotoxicosis and migraine). This is conceivable since oxidative stress exerted by ROS has been shown to significantly participate in the pathogenesis of hypertension and its related complications (i.e., IHD, CHF, RF, impotence, etc.,).

The production of NO is generally increased during atherosclerosis and hypertension. In addition to NO, these conditions are often referred to as oxidative stress-mediated diseases, where even higher increases in the production of superoxide and other ROS accompany the elevated production of NO. The eventual fate of NO is oxidation to nitrite (NO₂⁻) and nitrate (NO₃⁻), which are both end-products of NO metabolism under normal conditions. However, under oxidative stress conditions, besides the depletion of the natural antioxidant capacity, the major metabolic pathway of NO involves reaction with superoxide, resulting in the formation of a highly potent ROS, peroxynitrite. Peroxynitrite is an extremely hazardous ROS capable of interrupting many physiological functions. Recent evidence shows that, in vascular complications in diabetes, peroxynitrite rather than NO itself is responsible for the vascular disorders. Indeed, peroxynitrite is hundred times more potent than NO in causing some of the detrimental effects originally attributed to NO such as inhibition of cellular respiration through inactivation of critical mitochondrial enzymes.

Much progress has been made in our understanding of the role of the antioxidant enzymes, especially those involved in neutralizing superoxide (i.e., superoxide dismutase, SOD), in mediating the tissue resistance against oxidative stress and free radical injury.

In hypertension, for example, current therapies aim either to affect a certain system (e.g., rennin-angiotensin-aldosterone system, RAAS) or to target a specific receptor/s (beta-receptor, alpha-receptor, angiotensin receptor) to reduce elevated blood pressure. However, none of these therapeutic modalities have been shown to adequately affect the natural course of the disease or its outcome as evident by the still high incidence of morbidity and mortality associated with hypertension and its complications. This is conceivable since none of the current therapies address the multifactorial (multi-mediator) nature of the disease. In essence, however, many oxidative stress-mediated diseases like, for example, hypertension, can be described as a condition initiated by a yet unexplained hypersensitivity response of the vascular system to both endogenous and exogenous vasoconstrictors. As explained above, this simplified sequence of events leading to essential hypertension is accompanied by a significant increase of ROS production (oxidative stress) that is accompanied by decreased biological activity of the major vasodilator NO. Logically, therefore, for a candidate drug to be effective, it has to adequately address as many events as possible of this sequence.

The present invention is especially applicable in the treatment of conditions including, but not limited to, the ocular conditions, cardiovascular conditions and other conditions disclosed herein. As used herein, and as well-understood in the art, “treatment” is an approach for obtaining beneficial or desired results, including clinical results. For purposes of this invention, beneficial or desired clinical results can include one or more, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total). In particular embodiments, multifunctional ACE inhibitor compounds comprise Captopril or Enalapril.

These compounds enable various mechanisms of action, occurring simultaneously, at the required therapeutic sites. The anti-ROS activity of these compounds exert a significant impact on the severity, control, and the natural course of all vascular diseases involving oxidative-stress and free radical injury. The anti-superoxide activity of these compounds will reduce the effects of angiotensin at the site of drug action.

Formulations and Dosage

The compounds can be provided in a variety of formulations and dosages.

The compounds may be provided in a pharmaceutically acceptable form and/or in a salt form.

In one embodiment, the compounds are provided as non-toxic pharmaceutically acceptable salts. Suitable pharmaceutically acceptable salts of the compounds of this invention include acid addition salts such as those formed with hydrochloric acid, fumaric acid, p-toluenesulphonic acid, maleic acid, succinic acid, acetic acid, citric acid, tartaric acid, carbonic acid or phosphoric acid. Salts of amine groups may also comprise quaternary ammonium salts in which the amino nitrogen atom carries a suitable organic group such as an alkyl, alkenyl, alkynyl or aralkyl moiety. Furthermore, where the compounds of the invention carry an acidic moiety, suitable pharmaceutically acceptable salts thereof may include metal salts such as alkali metal salts, e.g., sodium or potassium salts; and alkaline earth metal salts, e.g., calcium or magnesium salts.

“Pharmaceutically acceptable salt” refers to any salt of a compound of this invention which retains its biological properties and which is not biologically or otherwise undesirable. Such salts may be derived from a variety of organic and inorganic counter-ions well known in the art and include, by way of example illustration, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The pharmaceutically acceptable salts of the present invention may be formed by conventional means, such as by reacting the free base form of the product with one or more equivalents of the appropriate acid in a solvent or medium in which the salt is insoluble, or in a solvent such as water which is removed in vacuo or by freeze drying or by exchanging the anions of an existing salt for another anion on a suitable ion exchange resin.

The present invention includes within its scope solvates of the multifunctional ACE inhibitor compounds and salts thereof, for example, hydrates.

The multifunctional compounds may have one or more asymmetric centers, and may accordingly exist both as enantiomers and as diastereoisomers. It is to be understood that all such isomers and mixtures thereof are encompassed within the scope of the present invention.

Additionally, all geometric isomers of the multifunctional ACE inhibitor compounds of formula I are included within the scope of this invention including, for example, all isomers with NO-donor and superoxide functionality.

The multifunctional ACE inhibitor compounds may be administered by oral, parenteral (e.g., intramuscular, intraperitoneal, intravenous, ICV, intracisternal injection or infusion, subcutaneous injection, or implant) may be formulated, alone or together, in suitable dosage unit formulations containing conventional non-toxic pharmaceutically acceptable carriers, adjuvants, excipients and vehicles appropriate for each route of administration. In addition to the treatment of pathology in humans the compounds of the invention may be effective in warm-blooded animals such as mice, rats, horses, cattle, sheep, dogs, cats, monkeys, etc.

The pharmaceutical compositions for the administration of the multifunctional ACE inhibitor compounds may conveniently be presented in dosage unit form and may be prepared by any of the methods well known in the art of pharmacy. The pharmaceutical compositions can be, for example, prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired therapeutic effect.

The pharmaceutical compositions containing the multifunctional ACE inhibitor compound as active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release. The pharmaceutical compositions of the invention may also be in the form of oil-in-water emulsions.

The present invention further provides use of the multifunctional ACE inhibitor compounds and functionalized ACE inhibitor compounds of the present invention in the manufacture of a medicament for the treatment of cardiovascular conditions involving ischemia, angina, hypertension, palpitations, arrhythmias (e.g., supraventricular, ventricular), cardiomyopathy, congestive heart failure, as well as other conditions for which the use ACE inhibiting agents have proven beneficial (e.g., diabetic nephropathy)

As described above, and as known in the art, pharmaceutical compositions for oral administration can take the form of bulk liquid solutions or suspensions, or bulk powders. More commonly, however, such compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, the nitrone compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

In some embodiments, the composition comprising multifunctional ACE inhibitor compounds where the ACE inhibitor component is Captopril or Lisinopril is formulated for oral administration.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent. Among the acceptable vehicles and solvents that may be employed are water, Ringer's solution and isotonic sodium chloride solution. The multifunctional ACE inhibitor compounds may also be administered in the form of suppositories for rectal administration of the drug.

A liquid formulation can be manufactured by dissolving the multifunctional ACE inhibitor compounds in a suitable solvent, such as water, at an appropriate pH, including buffers or other excipients.

As known by those of skill in the art, the preferred dosage of multifunctional ACE inhibitor compounds will depend on the age, weight, general health and severity of the respiratory condition of the individual being treated. Dosage may also need to be tailored to the sex of the individual. Dosage may also be tailored to individuals suffering from more than one condition or those individuals who have additional conditions which affect their general health and tolerance of treatment. Dosage, and frequency of administration of the multifunctional ACE inhibitor compound will also depend on whether the compounds are formulated for treatment of acute episodes of the condition or for the prophylactic treatment of the condition (e.g., as for migraines or anxiety). A skilled practitioner will be able to determine the optimal dose for a particular individual. Various formulations of the compounds and compositions described herein may be administered according to the variables described above. In particular, formulations for prophylactic treatment of a variety of conditions may be administered, daily, twice daily, thrice daily or four times daily and/or upon the occurrence of symptoms associated with the underlying condition. It is contemplated that individuals who are using a prophylactic formulation may on occasion need to administer doses in response to acute episodes of symptoms. Administration includes any of the methods or routes as described herein.

The multifuctional ACE inhibitor compounds as described herein may be administered to an individual in need thereof over a period of time consistent with treatment of the condition from which the individual suffers. In the case of periodic conditions, the treatment may be discontinued when the individual is no longer affected by the condition or deemed to be no longer in need of the treatment by a skilled practitioner. Examples of such time periods include days, weeks or months. Where the condition is a congenital or chronic condition such as certain cardiovascular conditions and others, it is envisioned that the treatment with the compounds described herein will be administered for a period of weeks, months, years or decades. The methods as described herein also include the administration of combinations of the multifunctional ACE inhibitor compounds as described herein, or combinations of the compounds described herein and other drugs used in the treatment of the cardiovascular conditions and other conditions described herein described herein or symptoms associated with these conditions.

As described in greater detail above, and as known by those of skill in the art, generally, the multifunctional ACE inhibitor compounds described herein are administered in a pharmaceutically effective amount. The amount of the multifunctional ACE inhibitor compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions comprising the multifunctional ACE inhibitor compounds described herein can be administered by any suitable routes including, by way of illustration, those described herein, such as, oral, topical via the eye, rectal, subcutaneous, intravenous, intramuscular, and the like. Depending on the intended route of delivery, the multifunctional ACE inhibitor compounds are preferably formulated as either oral or injectable compositions.

Injectable compositions are typically based upon injectable sterile saline or phosphate-buffered saline or other injectable carriers known in the art. As before, the multifunctional ACE inhibitor compound in such compositions is typically a minor component, often being from about 0.05 to 10% by weight with the remainder being the injectable carrier and the like.

The above-described components for orally administrable or injectable compositions are merely representative. Other materials as well as processing techniques and the like are set forth in Part 8 of Remington's Pharmaceutical Sciences, 18th edition, 1990, Mack Publishing Company, Easton, Pa., 18042, which is incorporated herein by reference.

The compounds of this invention can also be administered in sustained release forms or from sustained release drug delivery systems. A description of representative sustained release materials can be found in the incorporated materials in Remington's Pharmaceutical Sciences, supra.

Also provided are kits for administration of the multifunctional ACE inhibitor compound or composition comprising at least one multifunctional ACE inhibitor compound, that may include a dosage amount of at least one multifunctional ACE inhibitor compound or a composition comprising at least one multifunctional ACE inhibitor compound as disclosed herein. Kits may further comprise suitable packaging and/or instructions for use of the compound. Kits may also comprise a means for the delivery of the at least one multifunctional ACE inhibitor compound or compositions comprising at least one multifunctional ACE inhibitor compound, such as tube, or pressure pack for capsules, tablets, or other device as described herein.

In another aspect of the invention, kits for treating an individual who suffers from or is susceptible to cardiovascular conditions and other conditions described herein are provided, comprising a container comprising a dosage amount of an multifunctional ACE inhibitor compound or composition as disclosed herein, and instructions for use.

Kits may also be provided that contain sufficient dosages of the multifunctional ACE inhibitor compound or composition to provide effective treatment for an individual for an extended period, such as a week, 2 weeks, 3, weeks, 4 weeks, 6 weeks or 8 weeks or more.

All patents, patent applications and publications referred to herein are hereby incorporated herein by reference in their entirety.

The invention is further illustrated by the following nonlimiting examples.

### EXAMPLES

In the examples below, the following abbreviations have the following meanings. Abbreviations not defined below have their generally accepted meaning.

bpm=blood pressure in mmHg

dec=decomposed

dH₂O=distilled water

ELISA=enzyme-linked immuno-sorbent assay

EtOAc=ethyl acetate

EtOH=ethanol

g=gram

h=hour

Hz=hertz

i.v.=intravenous

L=liter

min=minutes

M=molar

MeOH=methanol

mg=milligram

MHz=megahertz

mL=milliliter

mmol=millimole

m.p.=melting point

N=normal

po=per os, oral

THF=tetrahydrofuran

t=time

tlc=thin layer chromatography

μg=microgram

μL=microliter

UV=ultraviolet

In the examples below, the temperatures are in degrees Celsius.

### Example 1

The approach to synthesis of compounds of Formula I is outlined in Scheme I below.

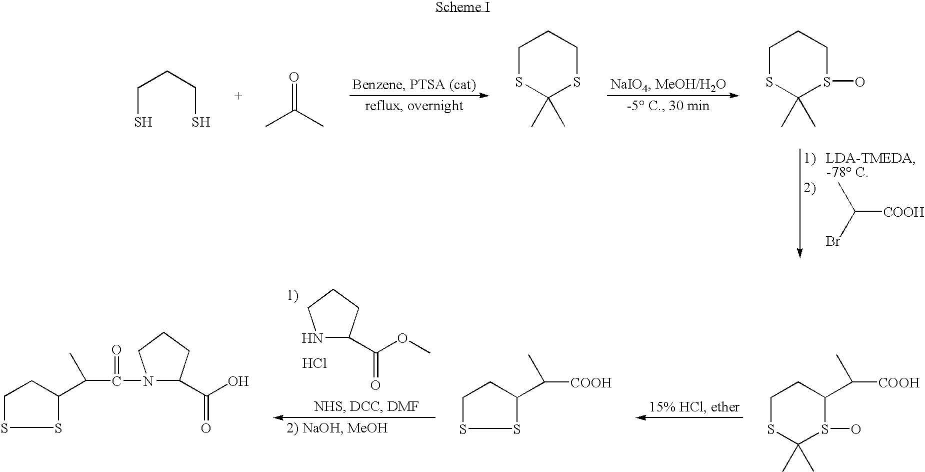

### 2,2,-dimethyl-1,3-dithiane (1)

1,3-propanedithiol (10.8 g, 0.1 Mol), acetone (6.4 g, 0.11 Mol) and a catalytic amount of para-toluenesulfonic acid in benzene (200 ml) were refluxed using a Dean-Stark apparatus to exclude water for 12 hours. The reaction mixture was cooled to room temperature and washed twice with 50 ml of 5% sodium hydroxide solution, water (50 ml) and brine (50 ml). The organic phase was dried over magnesium sulfate and evaporated to dryness. The residue was distilled at reduced pressure to afford 11.5 g of 2,2-dimethyl-1,3-dithiane as a colorless oil; b.p 86° C. at 20 mmHg.

### 2,2-dimethyl-1,3-dithiane-1-oxide (2)

2,2,-dimethyl-1,3-dithiane (4.32 g, 30 mmol) was dissolved in methanol (300 ml) and cooled to −5° C. in an ice-salt bath. Sodium metaperiodate (6.39 g, 30 mmol) in water (50 ml) was added dropwise to the vigorously stirred solution maintaining the internal temperature below 20° C. When the addition was complete, the reaction was stirred for further 30 min in the ice path until reaction was complete (1 hr). The reaction mixture was then filtered to remove the precipitated sodium periodate and the precipitate washed with chloroform and evaporated to near dryness on a water bath. The residue was extracted twice with 100 ml of dichloromethane. The organic phase was dried over sodium sulfate (if the organic phase was slightly colorized then activated charcoal can be used for decolorizing) and evaporated to dryness to give the title product as a colorless oil, which can be purified by column chromatography (ethyl acetate); b.p. 98-100° C. at 0.15 mmHg.

### 2-([1,2]Dithiolan-3-yl)-propionic acid (3)

A flame dried three necked flask equipped with pressure equalizing separatory funnel was charged with diisopropylamine (4.44 g, 41 mmol), dry tetrahydrofuran (THF, 10 ml) and cooled in a salt ice bath. n-Butyl lithium (2.5 M solution in hexane, 16 ml, 40 mmol) was added dropwise to the cooled solution of diisopropylamine. The reaction mixture was stirred for further 20 minutes in the ice bath and transferred to −78° C. path (dry ice-isopropanol). Freshly distilled N-N-N′-N′-tetramethyl ethylene diamine (4.64 g, 40 mmol) in 5 ml of dry THF was added dropwise to the LDA solution within 5 minutes and the reaction mixture was stirred for further 10 minutes at −78° C. 2,2-Dimethyl-1,3-dithiane-1-oxide (3.2 g, 20 mmol) in dry THF (10 ml) was added dropwise to the LDA-TMEDA complex and the reaction mixture was stirred for further 30 minutes. When the sulfoxide anion is already formed (usually a dark yellow to pale orange color is obtained), 2-bromo propionic acid (3.04 g, 20 mmol) in dry THF (10 ml) was added dropwise within 15 minutes and the reaction was stirred for another 8 hours before being quenched with 10 ml of 5 M HCl solution and left to warm to room temperature. The pH of the aqueous phase was checked to be acidic, and if necessary, more 1 N HCl solution was added to ensure acidity (pH=2-3) and the reaction was extracted with dichloromethane (2×100 ml). The organic phase was washed once with brine, dried over sodium sulfate and evaporated to dryness. Further purification was not necessary at this point and the crude product was subjected to hydrolysis in two phase system of ether and 15% aqueous HCl (1:1) for 12 hours at continued vigorous sting during which the organic phase turns to clear yellow. The organic phase is separated and washed with water and brine, evaporated to dryness and passed through a short column of silica gel then the product is recrystallized in ether-pentane. NMR (CDCl₃), 1.32 (d, 3H), 2.01 (m, 1H), 2.51 (m, 1H), 2.81 (m, 1H), 3.1 (m, 1H), 3.23 (m, 1H), 3.84 (m, 1H).

### N-{2-([1,2]Dithiolan-3-yl)-propionyl}-pyrrolidine-2-carboxylic acid (4)

2-([1,2]Dithiolan-3-yl)-propionic acid (0.534 g, 3 mmol), N-hydroxysuccinimide (0.345 g, 30 mmol) praline methyl ester hydrochloride (0.453 g, 3 mmol) and triethylamine (0.42 ml) were dissolved in dry dimethylformamide and cooled in the ice bath. DCC (0.618 g, 3 mmol) was dissolved in dry DMF and added dropwise with vigorous stirring to the reaction mixture and stirring was continued at room temperature for further 3 hours. The reaction was filtered and the precipitated DCU was washed with DMF. The DMF was evaporated to near dryness and the residue dissolved in dichloromethane and washed successively with water, 1N HCl, 5% NaHCO₃, water and brine. The organic phase was evaporated to dryness and the residue dissolved in methanol. To the methanol solution, 1N NaOH was added, and the reaction was left at room temperature for another 3 hours. The methanol was evaporated and the aqueous phase was washed twice with ether, and the organic phase was discarded. The aqueous phase was covered with a layer of ethyl acetate and acidified with hydrochloric acid. The organic phase was washed with brine, dried over magnesium sulfate, and evaporated to dryness. The crude product was purified by crystallization in benzene.

### Example 2

### Stereoselective synthesis of 2-([1,2]Dithiolan-3-yl)-propionic acid (5)

The stereo selective synthesis was achieved as described in the following scheme II:

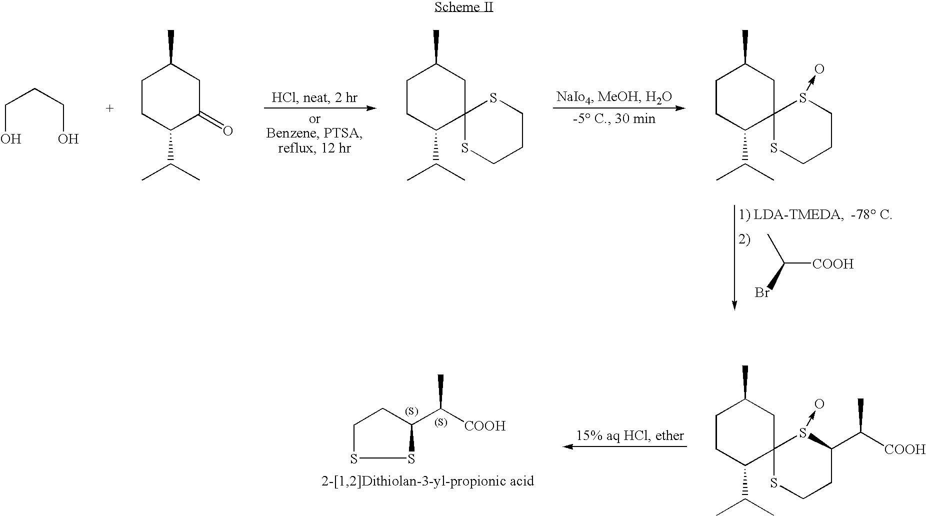

### Menthone trimethylenemercaptol (6)

This compound was obtained by two different procedures with good yields.

Method A: a mixture of 7.2 g of (−) or (+) menthone (46 mmol) and 5.2 g of 1,3-propanedithiol (47 mmol) was cooled in an ice bath and a stream of hydrogen chloride passed through the solution for two hours. After this time the mixture was quite turbid. Excess hydrogen chloride was removed in a vacuum desiccator over sodium hydroxide, and the mixture was dissolved in ether, washed with 5% sodium hydroxide solution, water and brine. The organic phase was dried over sodium sulfate and evaporated to dryness. The remaining oil was vacuum distilled (b.p 152-155° C., 3 mm). The distillate solidified and could be recrystallized from ethanol. Yield 6.5 g (58%), m.p. 41-42° C.

Method B: 7.2 g of (−) or (+) menthone (46 mmol) and 5.2 g of 1,3-propanedithiol (47 mmol) were dissolved in benzene (100 ml), and a catalytic amount of para-toluenesulfonic acid was added. The mixture was refluxed for 6 hours, and the water was excluded using a Dean-Stark apparatus. The reaction mixture was cooled and washed twice with 5% sodium hydroxide solution, water and brine. The organic phase was dried over sodium sulfate and evaporated to dryness. The residue was distilled at reduced pressure (b.p 152-155° C., 3 mm) to give an oil, which slowly solidifies and can be recrystallized from ethanol or isopropanol. Yield (59%)

### Menthone trimethylenemercaptol-S-oxide (7)

Menthone trimethylenemercaptol (4.88 g, 20 mmol) was dissolved in methanol (200 ml) and cooled to −5° C. in an ice-salt bath. Sodium metaperiodate (4.26 g, 30 mmol) in water (30 ml) was added dropwise to the vigorously stirred solution maintaining the internal temperature below 20° C. When the addition was complete the reaction was stirred for further 1 hr in the ice bath until reaction was complete. The reaction mixture was then filtered to remove the precipitated sodium periodate, and the precipitate was washed with dichloromethane. The filtrate was evaporated to near dryness on a water path. The residue was extracted twice with 100 ml of chloroform. If the organic phase was slightly colorized then activated charcoal can be used for decolorizing. The organic phase was dried over sodium sulfate and evaporated to dryness to give the title product as a white solid which can be purified by crystallization (dichloromethane-hexane) or column chromatography (ethyl acetate).

### 2-([1,2]Dithiolan-3-yl)-propionic acid (8)

A flame-dried three necked flask equipped with pressure equalizing separatory funnel was charged with diisopropylamine (4.44 g, 41 mmol), dry tetrahydrofuran (10 ml) and cooled in an ice bath. Butyl lithium (2.5 M solution in hexane, 16 ml, 40 mmol) was added dropwise to the cooled solution of diisopropylamine. The reaction mixture was stirred for further 20 minutes in the ice bath and transferred to −78° C. bath (dry ice-isopropanol). Freshly distilled N-N-N′-N′-tetramethyl ethylene diamine (4.64 g, 40 mmol) in 5 ml of dry THF was added dropwise to the LDA solution within 5 minutes and the reaction mixture was stirred for further 10 minutes at −78° C. Menthone trimethyl mercaptol S-oxide, + or −, (5.4 g, 20 mmol) in dry THF (20 ml) was added dropwise to the LDA-TMEDA complex and the reaction mixture was stirred for further 45 minutes. When the sulfoxide anion is already formed (usually a pale orange color is obtained), 2-bromo propionic acid (+ OR −, 3.04 g, 20 mmol) in dry THF (10 ml) was added dropwise within 15 minutes and the reaction was stirred for another 12 hours before being quenched with 10 ml of 5M HCl solution and left to warm to room temperature. The aqueous phase was checked to be acidic, and if necessary more 1N HCl was used to acidify the reaction mixture and then extracted with dichloromethane (2×100 ml). The organic phase was washed with water and brine, dried over sodium sulfate and evaporated to dryness. Further purification was not necessary at this point and the crude product was subjected to hydrolysis in two phase system of ether and 15% aqueous HCl (1:1) overnight at continued vigorous stirring during which the organic phase turns to clear yellow. The organic phase was separated and washed with water and brine and evaporated to dryness. The product was passed through a short column of silica gel and eluted with dichloromethane followed by 20% ethyl acetate in dichloromethane, then the product was recrystallized in dichloromethane-pentane or ether-hexane.

This method was utilized to prepare different enantiomers of 2-[1,2] Dithiolan-3-yl-propionic acid, that were coupled with proline methyl ester hydrochloride in the same manner that was mentioned above. The final saponification of the obtained esters afforded analogues of Formula I.

### Example 3

Examples for the Synthesis of Formula III Analogues

### Synthesis of N-{2-[4-([1,2]Dithiolan-3-yl)-1-ethoxycarbonyl-butylamino]-propionyl}-pyrrolidine-2-carboxylic acid

This compound was synthesized as described in the following scheme III:

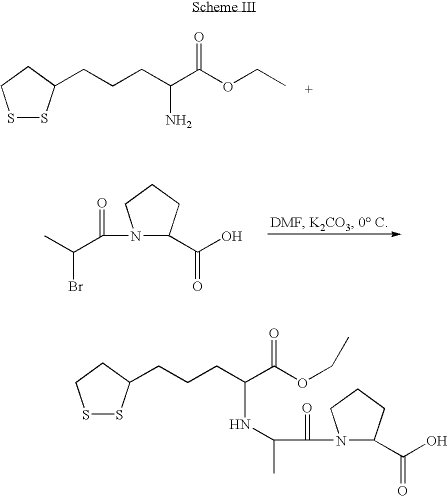

α-amino ethyl lipoate was reacted with 2-bromo propionyl proline in DMF in the presence of potassium carbonate to afford the title product.

The following scheme describes the synthesis of 2-bromo propionyl bromide:

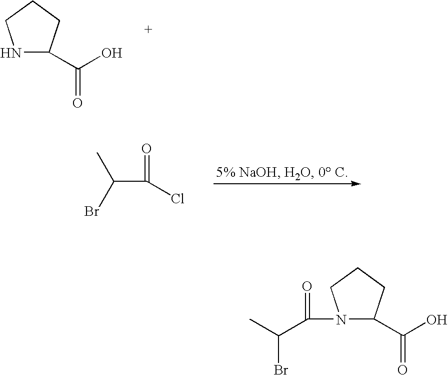

This compound was synthesized by reacting 2-bromopropionyl chloride with proline in 5% NaOH solution at −5° C. for 2 hours. After acidification with 1N sulfuric acid and extraction with ethyl acetate the product was recrystallized with ethyl acetate-petroleum ether.
α-Amino ethyl lipoate was synthesized as described in the following scheme IV:

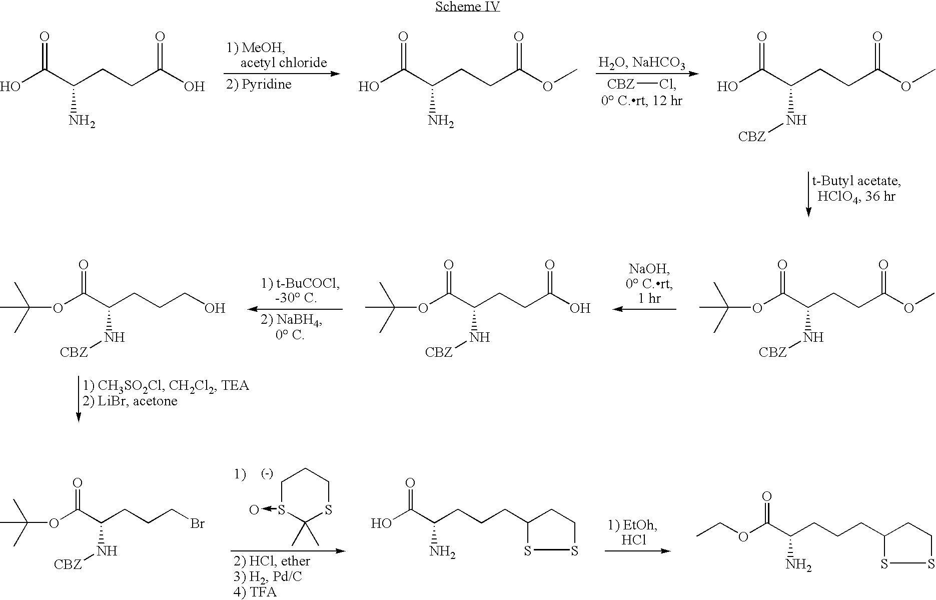

### 2-Amino ethyl lipoate

### Glutamic acid-5-methyl ester (9)

Acetyl chloride (20 ml) was added to methanol (300 ml) and the solution was cooled in an ice bath then L-glutamic acid (36 g) was added and the solution was stirred until all the solid has dissolved, and the reaction was kept at room temperature for 20 hours. Dry pyridine (40 ml) was added, and the reaction mixture was kept at room temperature for another 16 hours. The precipitated product (20 g, 50%) was filtered and, washed successively with ether and air dried. The product, glutamic acid 5-methyl ester, can be used directly for the next step. An analytical sample can be obtained by recrystallization in 70% aqueous methanol.

### N-Carbobenzyloxy-glutamic acid 5-methyl ester (10)

Glutamic acid 5-meythyl ester (16.1 g, 0.1 mol) and sodium hydrogen carbonate (16.8 g, 0.2 mol) were dissolved in 200 ml of water and cooled in an ice bath. Benzyl chloroformate (17 g, 0.11 mol) was added dropwise with vigorous stirring. When the addition was complete the reaction mixture was stirred overnight at room temperature. The reaction was extracted once with ethyl acetate and the organic phase was discarded, and the aqueous phase was covered with a layer of ethyl acetate and acidified with 1 N HCl to pH 1-2. The ethyl acetate layer was separated, and the aqueous phase was extracted twice with ethyl acetate. The organic extracts were pooled, washed with brine, dried over sodium sulfate and evaporated to dryness to afford oil, which solidifies upon triturating with petroleum ether.

### N-CBZ-5-methyl-t-butyl glutamate (11)

N-CBZ-glutamic acid 5-methyl ester (25 g, 0.1 mol) was dissolved in t-butyl acetate (250 ml) and 70% perchloric acid (1.5 ml) was added. The solution was stoppered, stirred and left to stand at room temperature for 48 hours. The flask was opened carefully and added dropwise to a saturated solution of NaHCO₃. When the addition was complete, the mixture was extracted with ethyl acetate and the organic phase washed with water and brine, dried over magnesium sulfate and evaporated to dryness. The oily product was purified by column chromatography over silica gel eluted with hexane ethyl acetate (4:1).

### 2-Benzyloxycarbonylamino-pentanedioic acid 1-tert-butyl ester (12)

N-CBZ-5-methyl-t-butyl glutamate (35.1 g, 0.1 mol) was dissolved in 200 ml of methanol, stirred and cooled in an ice bath. 1N Solution of sodium hydroxide (100 ml, 0.1 mol) was added dropwise and the resulting solution was stirred at room temperature for further 1 hour. Methanol was evaporated on a rotary evaporator and the remaining aqueous solution extracted once with ethyl acetate, and the organic phase was discarded, and the aqueous phase acidified with 10% citric acid solution and extracted twice with ethyl acetate. The ethyl acetate was evaporated to dryness to afford the title compound as colorless oil.

### 2-Benzyloxycarbonylamino-5-hydroxy-pentanoic acid tert-butyl ester (13)

2-Benzyloxycarbonylamino-pentanedioic acid l-tert-butyl ester, 10 mmol, was dissolved in 30 ml of dry tetrahydrofuran and cooled to −40° C. in dry ice isopropanol bath. Triethylamine (12 mmol) was added, followed by slow addition of isobutyl chloroformate and the reaction mixture was stirred below −20° C. for 45 minutes. The precipitated triethylamine hydrochloride was filtered off and washed with THF (cooled in dry ice), and the filtrate was added as quickly as possible to the suspension of sodium borohydride in 20 ml of THF-water (8:1) at 0° C. with vigorous stirring and left at room temperature for further 3 hours. The reaction mixture was acidified with dilute HCl to pH 5 and the THF was removed under vacuum on a rotary evaporator, and the water was extracted with ethyl acetate and the extracts were washed with water, brine and dried with sodium sulfate and evaporated to dryness. The product was purified by column chromatography on silica gel, eluting with hexane-dichloromethane (4:1), to give colorless oil. Yield 89%.

### 2-Benzyloxycarbonylamino-5-bromo-pentanoic acid tert-butyl ester (14)

To a solution of 10 mmol of 2-benzyloxycarbonylamino-5-hydroxy-pentanoic acid tert-butyl ester and 20 mmol of CBr₄ in 120 ml of THF, triphenylphosphine was added in one portion of 20 mmol, and the reaction mixture was stirred overnight. The THF was removed under reduced pressure and the residue was chromatographed on silica gel column eluting with hexane-ethyl acetate (9:1) to afford the title product.

Alternatively 2-amino lipoic acid can be synthesized from pyroglutamic acid as described in the following scheme V:

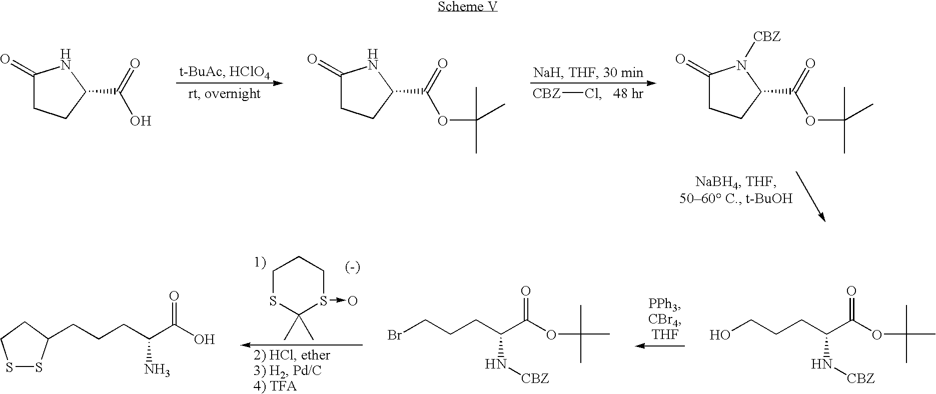

### t-Butyl pyroglutamate (15)

To a suspension of S-(−)-pyroglutamic acid (12.9 g, 0.1 mol) in 200 ml of t-butyl acetate, 70% perchloric acid (3 ml, 0.11 mol) was added and the reaction mixture was stirred overnight in a tightly closed flask. Then the reaction mixture was slowly poured into a saturated solution of NaHCO₃ and the product was extracted with ether. The organic phase was washed with brine, dried over magnesium sulfate and evaporated to dryness to provide 11.3 g (60% yield) of t-butyl L-pyroglutamate. An analytical sample can be obtained by crystallization in ether-hexane, m.p. 91-92° C.

### N-CBZ-t-Butyl pyroglutamate (16)

A dry two necked flask, flushed with dry nitrogen and protected with calcium chloride guard tube, was charged with anhydrous tetrahydrofuran (50 ml) and t-butyl pyroglutamate (1.86 g, 10 mmol) and cooled in an ice bath. Sodium hydride (0.73 g, 60% suspension in paraffin oil, 11 mmol) was added to the reaction flask in portions and the reaction mixture was left to stir at room temperature for 30 min. Benzyl chloroformate (1.87 g, 11 mmol) dissolved in anhydrous THF was added dropwise and after the addition was complete the reaction mixture was stirred at room temperature for 48 hours. The solvent was removed under reduced pressure and the residue was extracted with ethyl acetate and washed with 5% citric acid solution, water and brine. The organic phase was dried with magnesium sulfate and evaporated to dryness. The crude product was chromatographed on silica gel (hexane-ethyl acetate 3:1) to afford the title product as a white solid (yield 78%, m.p 43-45° C.).

### 2-Benzyloxycarbonylamino-5-hydroxy-pentanoic acid tert-butyl ester (17)

To a solution of 1 mmol of N-CBZ-t-Butyl pyroglutamate and 1 mmol of NaBH₄ in 25 ml of anhydrous THF, 5 ml of tert-butyl alcohol in 5 ml of anhydrous THF was added at 50-60° C. dropwise within 30 min. The reaction was continued 10 minutes longer and the solvent was removed under reduced pressure. To the residue, 10% citric acid solution was added, and the product was extracted with ethyl acetate, washed with water and brine, dried over anhydrous magnesium sulfate and filtered. The ethyl acetate was removed under reduced pressure and the crude product was purified on silica gel column with dichloromethane-ethyl acetate (4:1) as the eluent to provide the title product as colorless oil (yield 56%).

### Example 4

Synthesis of Formula IV Compounds

The multifunctional ACE inhibitor compounds of Formula IV can be prepared from readily available starting materials using the following general methods and procedures, Scheme VI, VII, and VIII.

As illustrated in Scheme VI, L-lysine reacts with N-benzyloxycarbonyloxy-5-norborene-2,3-dicarboximide to provide N⁶-(benzyloxycarbonyl)-L-lysine (1). The N²-amino group is protected with tert-butoxycarbonyl using di-tert-butyl dicarbonate to give the fully protected L-lysine (2), which condenses with L-proline tert-butyl ester in the presence of N,N¹-dicyclohexylcarbodiimide to generate N-[N²-(tert-butoxycarbonyl)-N⁶-(benzyloxycarbonyl)-L-lysyl]-L-proline tert-butyl ester (3). The tert-butoxycarbonyl and tert-butyl ester protecting groups in compound (3) can be 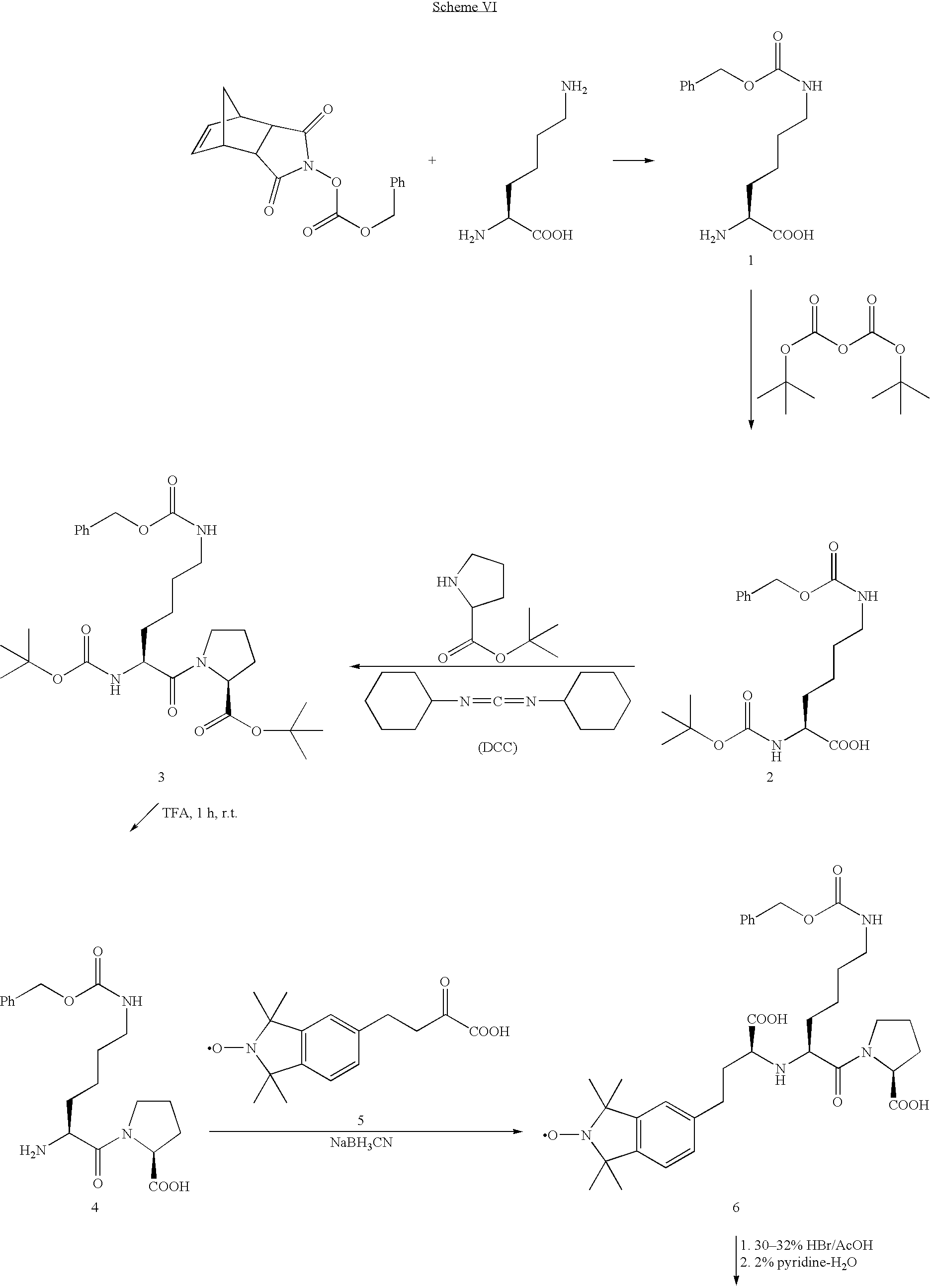 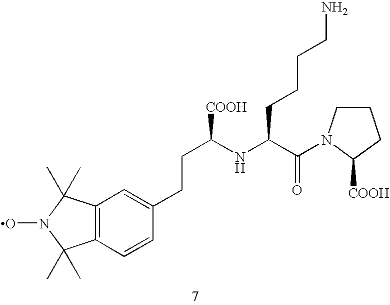
removed by treating compound (3) with trifluoroacetic acid providing N-[N⁶-(benzyloxycarbonyl)-L-lysyl]-L-proline (4). Reductive coupling of (4) with 2-oxo-4-(2-oxy-1,1,3,3-tetramethyl-2,3-dihydro-1H-isoindol-5-yl)butyric acid (5) from Scheme 2 using sodium cyanoborohydride yields N-{N²-[1(S)-carboxy-3-(2-oxy-1,1,3,3-tetramethyl-2,3-dihydro-1H-isoindol-5-yl)propyl]-N⁶-benzyloxycarbo nyl-L-lysyl}-L-proline (6). Removal of the benzyloxycarbonyl group is achieved by treating compound (6) with 30-32% HBr in glacial acetic acid and then 2% pyridine/H2O solution generating the final product N-{N²-[1(S)-carboxy-3-(2-oxy-1,1,3,3-tetramethyl-2,3-dihydro-1H-isoindol-5-yl)propyl]-L-lysyl}-L-proline (7).

The nitroxide (5), 2-oxo-4-(2-oxy-1,1,3,3-tetramethyl-2,3-dihydro-1H-isoindol-5-yl)butyric acid, may be synthesized by the method in Scheme 2:

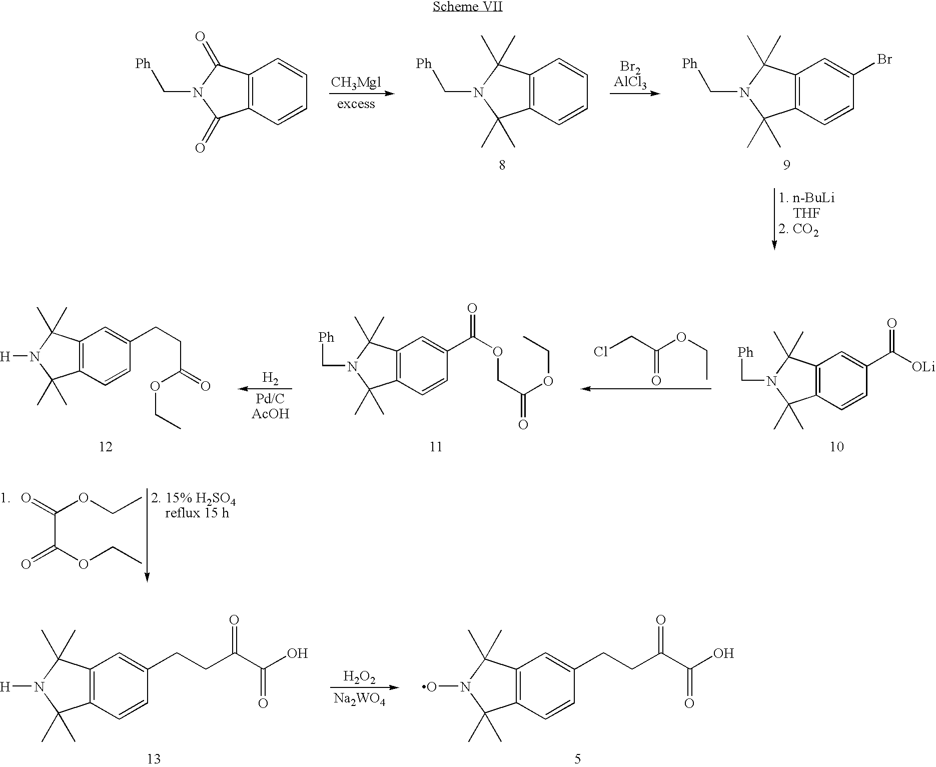

The commercially available starting material N-benzylphthalimide is treated with more than 4-fold methylmagnesium iodide to produce 2-benzyl-1,1,3,3-tetramethyl-2,3-dihydro-1H-isoindoline (8), which is brominated to yield 2-benzyl-5-bromo-1,1,3,3-tetramethyl-2,3-dihydro-1H-isoindoline (9). The bromo compound reacts with n-butyllithium and then carbon dioxide to give the corresponding carboxylic lithium salt (10) that is treated with ethyl chloroacetate to generate 2-benzyl-1,1,3,3-tetramethyl-2,3-dihydro-1H-isoindoline-5-carboxylic acid ethoxycarbonylmethyl ester (11). Hydrogenation of this ester provides 3-(1,1,3,3-tetramethyl-2,3-dihydro-1H-isoindol-5-yl)propionic acid ethyl ester (12), which is treated with oxalic acid diethyl ester in the presence of sodium methoxide and then sulfuric acid to afford 2-oxo-4-(1,1,3,3-tetramethyl-2,3-dihydro-1H-isoindol-5-yl)butyric acid (13). Oxidation of compound (13) produces the nitroxide 2-oxo-4-(2-oxy-1,1,3,3-tetramethyl-2,3-dihydro-1H-isoindol-5-yl)butyric acid (5).

Scheme VIII illustrates the methodology for the synthesis of N-{N²-[1(S)-carboxy-3-(2-oxy-1,1,3,3-tetramethyl-2,3-dihydro-1H-isoindol-5-yl)propyl]-L-lysyl}-L-pyrrolidine-2-methylene nitrate (18). The synthesis starts with the reaction between N²-(tert-butoxycarbonyl)-N-6-(benzyloxycarbonyl)-L-lysine (2) and (S)-2-(tert-butoxycarbonyloxy-methyl)pyrrolidine to give N-[N²-(tert-butoxycarbonyl)-N⁶-(benzyloxycarbonyl)-L-lysyl]-(S)-2-(tert-butoxycarbonyloxy-ethyl)pyrrolidine (14). The tert-butoxycarbonyl protecting groups in compound (14) can be removed by treating compound (14) with trifluoroacetic acid providing N-[N⁶-(benzyloxycarbonyl)-L-lysyl]-(S)-2-pyrrolidinemethanol 15, which is reductively coupled with 2-oxo-4-(2-oxy-1,1,3,3-tetramethyl-2,3-dihydro-1H-isoindol-5-yl)butyric acid (5) using sodium cyanoborohydride giving N-{N²-[1 (S)-carboxy-3-(2-oxy-1,1,3,3-tetramethyl-2,3-dihydro-1H-isoindol-5-yl)propyl]-N⁶-benzyloxy-carbonyl-L-lysyl}-(S)-2-pyrrolidinemethanol (16). The amino group in compound (16) is protected by using di-tert-butyl dicarbonate to generate N-{N²-[1(S)-carboxy-3-(2-oxy-1,1,3,3-tetramethyl-2,3-dihydro-1H-isoindol-5-yl)propyl]-N²-(tert-butoxycarbonyl)-N⁶-benzyloxycarbonyl-L-lysyl}-(S)-2-pyrrolidinemethanol (17), which is nitrated and deprotected to produce the final nitrate product (18).

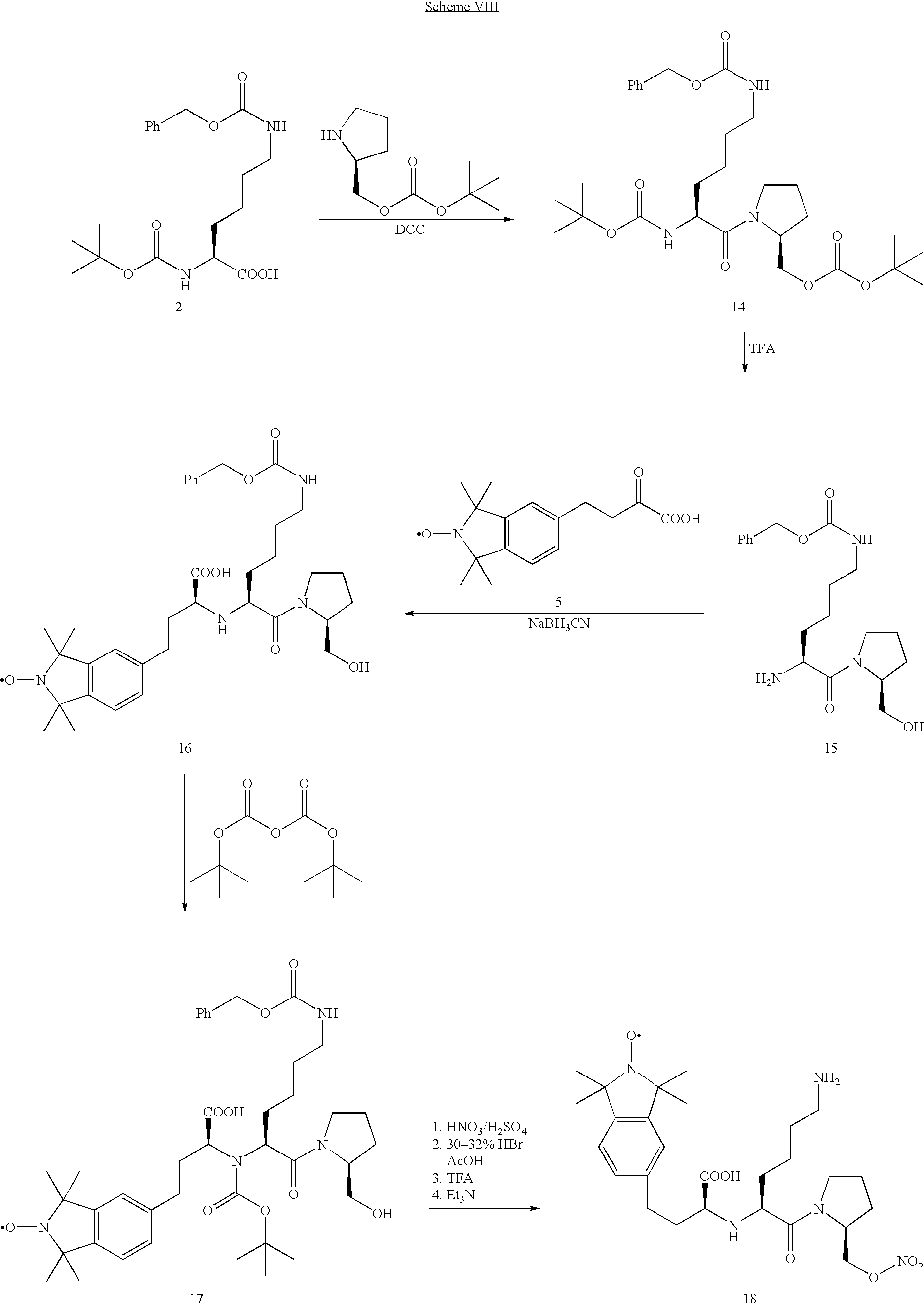

Compound (19) may be synthesized by methodologies similar to that as described in Scheme VIII by using 2-oxo-4-phenylbutyric acid to replace compound (5). Compound (20) can be obtained by synthetic methodologies similar to that as described in Scheme VI by using 4-(3H-benzo[1,2]dithiol-5-yl)-2-oxobutyric acid to replace compound (5). Compound (21) may be synthesized by methodologies similar to that as described in Scheme VIII by using 4-(3H-benzo[1,2]dithiol-5-yl)-2-oxobutyric acid to replace compound (5).

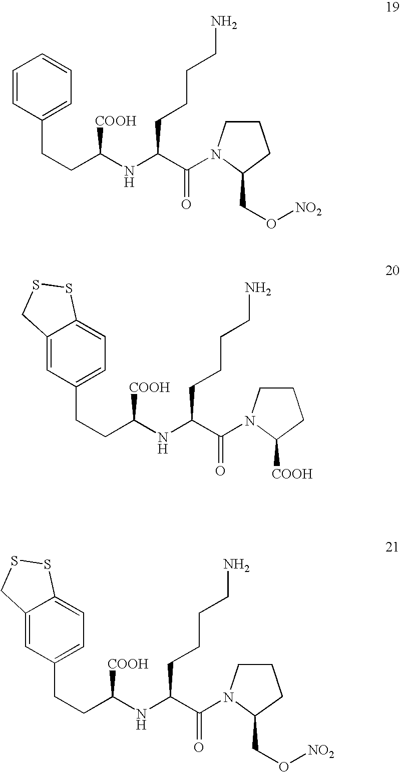

### Example 5

Measurement of NO-Donating Properties

(a) NO-Induced Formation of cGMP:

Nitric oxide (NO) stimulates guanylate cyclase in smooth muscle cells, which results in the formation of cyclic GMP. Primary cultures of rat aortic smooth muscle cells (RAOSMC) is used to measure cyclic GMP generated in the presence of NO-donors. Primary cultures (passage 2) of RAOSMC are purchased from Cell Applications and grown in 6-well dishes (9.5 cm²; Costar) in growth media of DMEM/F12 (1:1) supplemented with 10% fetal bovine serum (Hyclone), 2 mM L-glutamine, 100 U/ml penicillin, and 100 μg/ml streptomycin in a 37° C. incubator in an atmosphere of 5% CO₂-95% air. Cells are assayed at 90% confluency. At the time of assay, growth media is replaced with warm (37° C.) assay buffer (Hanks BSS containing 10 mM Hepes and 0.1% BSA, pH 7.5). The phophodiesterase inhibitor zaprinast (30 μM; Calbiochem) is added for 15 minutes prior to addition of test drug. The test drug is added and the cells incubated at 37° C. for 15 min. The assay is stopped by aspiration of assay buffer, and addition of 0.4 ml of cold 0.1 M HCl. The dishes are incubated for 15 min at 4° C., and the cell lysate scraped and transferred to a microfuge tube on ice. The tube is centrifuged for 4 min at 12,000 RPM at 4° C., and the supernatant assayed for cyclic GMP content by ELISA using the acetylation protocol as described by the kit manufacturer (Assay Designs, Ann Arbor, Mich.).

(b) Measurement of Nitrate and Nitrite Formed from NO:

Nitric oxide (NO) is rapidly converted to nitrate and nitrite in aqueous solution. Subsequent enzymatic conversion of nitrate to nitrite, followed by colorimetric determination of nitrite concentrations, is used to determine the amount of NO produced in solution. This assay will measure the production of NO by test compounds in the presence of cells which can metabolize organic nitrates to NO. Cells used is primary cultures of RAOSMC (see above). Cells (passage 3-6) are grown in 24-well dishes (1.9 cm²; Costar) in growth media of 0.5 ml DMEM/F12 (1:1) supplemented with 10% fetal bovine serum (Hyclone), 2 mM L-glutamine, 100 U/ml penicillin, and 100 μg/ml streptomycin in an atmosphere of 5% CO₂-95% air at 37° C. Cells are assayed at 90% confluency. Test compounds are added to the media, and the cells incubated for 2-24 hours in an atmosphere of 5% CO₂-95% air at 37° C. At the end of this incubation, the assay media is collected and assayed for NO production. Nitrate is converted to nitrite by adding 10 μl of nitrate reductase (1 U/ml) and 10 μl of NADH (2 mM) to 80 μl of assay media or standard, which is then incubated for 30 min at 37° C. Nitrite is quantitated by adding 100 μl of Greiss reagent (1:1 mixture of Greiss reagent I and Greiss reagent II prepared just before assay) and measuring optical density at 550 μm. Standards of sodium nitrate and sodium nitrite (1-100 μM) are made in growth media and processed as described for cell media samples.

(c) Measurement of NO-Induced Relaxation of Rat Aorta:

Nitric oxide (NO) induces the rapid relaxation of precontracted vascular smooth muscle, and this assay is used to measure NO generated from test compounds in the presence of blood vessels. Male Sprague-Dawley rats (200-250 g) are purchased from Comparative Biosciences (Mountain View, Calif.) and used in the rat aortic rings relaxation studies in a tissue bath preparation. Thoracic aorta are removed following anesthesia with i.p. injection of ketamine (50 mg/kg) and xylazine (10 mg/ml). The adventia surrounding the vessel is carefully removed, and the aorta is cut into rings of 4-5 mm and mounted in the tissue bath (5 ml volume). Kreb's-Henseleit buffer is used as the tissue bath buffer, and it is constantly gassed with carbogen and maintained at 37° C. The rings are preloaded with 2 g tension and equilibrated for 90 min with buffer changed every 15 min. After stabilization, the rings are contracted with phenylephrine (PE; 0.3 μM). Dose-response curves for the relaxation of PE-contracted rings are performed by cumulative addition of test drug. After the last addition of test drug, sodium nitroprusside (1 μM) is added to induce complete relaxation of the ring. Values are expressed as the percent of maximal tension induced by 0.3 μM PE.

### Example 6

Measurement of SOD-Mimetic Properties

Lucigenin is an acridylium dinitrate compound that emits light on reduction and interaction with the superoxide anion (O₂⁻), and is used to measure O₂⁻ production. Compounds are tested for their ability to scavenge O₂⁻ generated by the reaction of xanthine+xanthine oxidase. Reduction of the lucigenin chemiluminescence signal in the presence of xanthine+xanthine oxidase is used as the measurement of O₂⁻ scavenging potency. The assay reaction buffer is Hank's BSS containing 20 μM Hepes (pH 7.4), 0.1% BSA, 250 μM lucigenin, 200 μM xanthine, and test compound. A vial containing 1.6 ml of the reaction mixture and 0.2 ml of test compound is placed in a liquid scintillation counter and dark adapted for 5 min. The reaction is started by addition of 0.2 ml of xanthine oxidase (0.0005 U/ml final), and emitted light is recorded continuously for 10 min. Superoxide dismutase (SOD; 0.5 U/ml final) is used as a positive control to completely inhibit the SO-specific signal. The light signal (cpm) at 5 minutes is used to compute the percent reduction of control response.

### Example 7

Measurement of Anti-Hypertensive Properties In Vivo

The anti-hypertensive effect of the multi-functional compounds are assessed according to a method modified from that described by H. Gerhard Vogel Ed 1997 (In: Drug Discovery and Evaluation—Pharmacological assays; Chapter A.1.3; Springer Verlag). Briefly, male Sprague-Dawley rats (200-250 g; purchased from Comparative Biosciences, Mountain View, Calif.) are anaesthetized with thiopentone sodium (120 mg/kg, i.p), trachea is cannulated to facilitate spontaneous respiration and the rectal temperature is maintained at 37° C. with a homeothermic blanket system (Harvard Apparatus, Holliston, Mass.). The right carotid artery is cannulated and connected to a pressure transducer (SensNor 840, Horten, Norway) for the measurement of arterial blood pressure (systolic, diastolic, mean arterial) and heart rate which are recorded for the duration of the experiment and displayed on a PowerLab 8 recording system (AD Instruments, Colorado, USA). The left jugular vein is cannulated for the administration of drugs. The response to drugs are quantified as either absolute change in blood pressure (mmHg) (or heart rate, bpm (beats per minute)) or area under the response curve (mean arterial blood pressure, mmHg.min) using the chart analysis software. When stable homodynamic conditions are achieved for at least 30 min, increasing bolus i.v. doses of angiotensin I (0.01 mg/kg, 0.10 mg/kg, 1 mg/kg, 10 mg/kg) are administered after injection of test compounds (1,10, or 30 mg/kg, i.v.), standard non-fucntionalised ACE inhibitor compound (e.g. lisinopril, 1, 10, or 30 mg/kg, i.v.) or the appropriate vehicle (injected 15 min before injecting angiotensin I. The activity of the test compound was compared against the non-functionalised ACE inhibitor and vehicle in affecting the blood pressure responses to angiotensin I.

### Example 8

Protocol for Measuring ACE Activity

Angiotensin converting enzyme (ACE) is a dipeptidyl carboxypeptidase that releases the C-terminal dipeptide His-Leu from decapeptide angiotensin I, converting it to the vasoconstrictor angiotensin II (Ang II). The assay for ACE activity will measure the cleavage of a fluorogenic peptide substrate, (7-methoxycoumarin-4-yl)acetyl-Arg-Pro-Pro-Gly-Phe-Ser-Ala-Phe-Lys(2,4 dinitrophenyl; R&D Systems). This peptide substrate contains a fluorescent 7-methoxycoumarin group that is internally quenched by resonance energy transfer to the 2,4-dinitrophenyl group. ACE cleaves the peptide between the Ala-Phe (between the fluorescent group and the quencher group), resulting in an increase in fluorescence. The assay is carried out in a 96-well plate. To each well is added the test compound, fluorogenic substrate (10 μM), and recombinant human somatic ACE (10 ng; R&D Systems) in 50 mM sodium borate buffer, pH 8.2, in a final volume of 100 μl. Fluorescence changes (relative fluorescence units/min, RFU/min) are measured using a fₘₐₓ fluorescence microplate reader (Molecular Devices with SoftmaxPro software) at 37° C. with excitation and emission wavelengths of 320 nm and 405 nm, respectively. A background rate determined for samples containing no ACE is subtracted from all reactions to calculate the initial rates in RFU/min. To determine IC₅₀ values of test compounds, initial rate data are plotted as percentage activity relative to uninhibited control reactions versus inhibitor concentration. Reference compounds used will be the ACE inhibitors Quinapril and Lisinopril.

### Example 9

Biological Properties of a Multifunctional ACE Inhibitor

The ACE activity of the compound N-{2-([1,2]dithiolan-3-yl)-propionyl}-pyrrolidine-2-carboxylic acid was measured as described above. The value of IC₅₀ was found 64 μM.

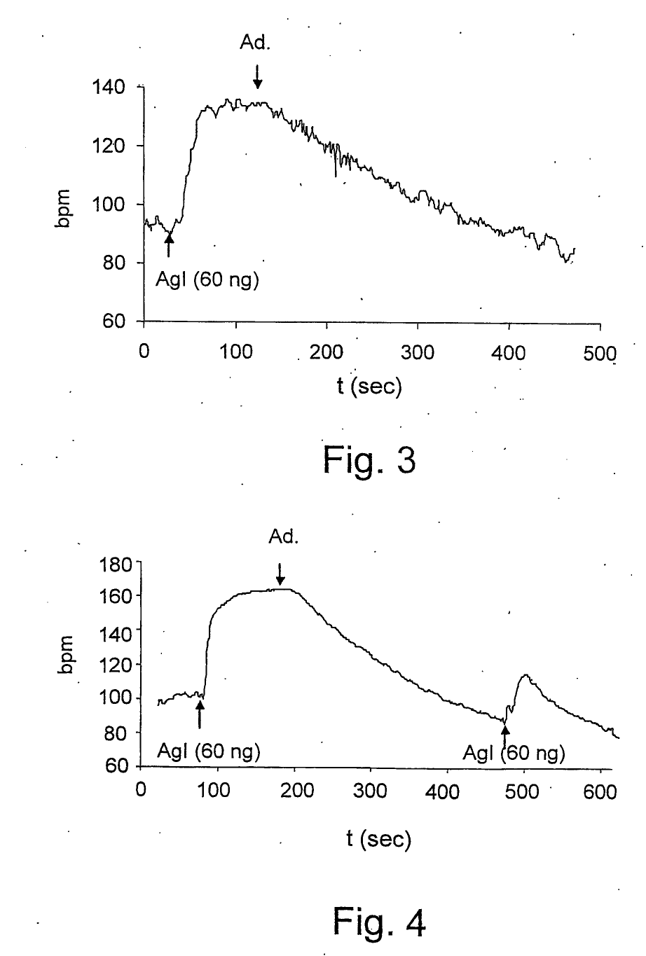

The antihypertensive properties of the said compound was characterized by injecting it (50 μM) intravenously to healthy SD rats following i.v. injection of 60 ng of angiotensin-I (AgI). FIG. 3 shows the blood pressure in mm Hg (bpm) as the functions of time, the time of injection being marked as “Ad”. FIG. 4 shows another experiment, the same conditions, followed by reinjection of 60 ng AgI after the return of the blood pressure to normal.

### Example 10

Pharmaceutical Formulations of Multifunctional ACE Inhibitor Compounds

The following formulations illustrate representative pharmaceutical compositions comprising multifunctional ACE inhibitor compounds. These formulations are, however, illustrative and are not intended to limit the invention as claimed.

Formulation 1—Tablets

A multifunctional ACE inhibitor compound is admixed as a dry powder with a dry gelatin binder in an approximate 1:2 weight ratio. A minor amount of magnesium stearate is added as a lubricant. The mixture is formed into 240-270 mg tablets (80-90 mg of active nitrone compound per tablet) in a tablet press.

Formulation 2—Capsules

A multifunctional ACE inhibitor compound is admixed as a dry powder with a starch diluent in an approximate 1:1 weight ratio. The mixture is filled into 250 mg capsules (125 mg of active nitrone compound per capsule).

Formulation 3—Liquid

A multifunctional ACE inhibitor compound (125 mg), sucrose (1.75 g) and xanthan gum (4 mg) are blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color are diluted with water and added with stirring. Sufficient water is then added to produce a total volume of 5 mL.

Formulation 4—Injection

The multifunctional ACE inhibitor compound is dissolved in a buffered sterile saline injectable aqueous medium to a concentration of approximately 5 mg/mL.

Formulation 5—Ointment

Stearyl alcohol (250 g) and white petroleum (250 g) are melted at about 75° C. and then a mixture of a multifunctional ACE inhibitor compound (50 g), methylparaben (0.25 g), propylparaben (0.15 g), sodium lauryl sulfate (10 g), and propylene glycol (120 g) dissolved in water (about 370 g) is added and the resulting mixture is stirred until it congeals.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof. The preceding examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

1-48. (canceled)

49. A method of treating or preventing a disorder selected from the group consisting of disorders in which treatment with an ACE-inhibitor is indicated, cardiovascular disorders, renal disorders, and diabetes associated disorders, in a mammal in need of said treating or preventing, comprising administering to said mammal an effective amount of a multifunctional ACE-inhibitor comprising in one molecule
i) an ACE-inhibitor component;
ii) at least one reactive oxygen species (ROS) scavenger component, not identical with said ACE-inhibitor component; and optionally,
iii) at least one nitric oxide (NO) donor component, not identical with said ROS scavenger component.

50. A method according to claim 49, wherein said multifunctional ACE-inhibitor comprises
i) an ACE-inhibitor component;
ii) at least one ROS-scavenger component not identical with said ACE-inhibitor component; and
iii) at least one nitric oxide (NO) donor component, not identical with said ROS scavenger component.

51. A method according to claim 49, wherein said ACE-inhibitor component is selected from the group consisting of compounds used in medicine as ACE-inhibitors, derivatives thereof, and compounds exhibiting affinity for ACE.

52. A method according to claim 49, wherein said ROS-scavenger component comprises an antioxidant reacting with an ROS selected from the group consisting of superoxide, hydroxyl radicals, peroxynitrite, and hypochlorite.

53. A method according to claim 49, wherein said ROS-scavenger component comprises an alkenyl group, aryl group, substituted aryl group, sulfhydryl, dithiol in oxidized or reduced form, or a group that is converted in vivo into a sulfhydryl in its oxidized or reduced form.

54. A method according to claim 49, wherein said ROS-scavenger component comprises a substituted N-oxide free radical, or a substituted or unsubstituted lipoic acid moiety,

55. A method according to claim 49, wherein said ROS-scavenger component comprises an N-oxide free radical, wherein the nitrogen of said N-oxide free radical is within a 3-, 4-, 5-, 6- or 7-membered ring, and wherein the ring may be substituted or unsubstituted with straight or branched alkyl groups, alkoxy groups or groups capable of donating NO.

56. A method according to claim 49, wherein said NO-donor comprises a group capable of providing nitric oxide in a form selected from uncharged and charged.

57. A method according to claim 49, wherein said NO-donor component comprises a group selected from —ONO₂, —ONO, —SNO, and —NONOate.

58. A method according to claim 49, wherein said ACE-inhibitor component is derived from an ACE-inhibitor selected from the group consisting of Alacepril, Benazepril, Captopril, Ceronapril, Cilazapril, Delapril, Enalapril, Enalaprilat, Fosinopril, Imidapril, Lisinopril, Moveltopril, Perindopril, Quinapril, Ramipril, Spirapril, Temocapril, and Trandolapril.

59. A method according to claim 49, wherein said multifunctional ACE-inhibitor has Formula I: 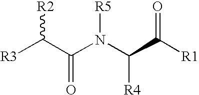
wherein R¹ may be selected from H, OH, NH₂, and alkoxy;
R² may be selected from —H and lower alkyl;
R³ may be selected from -alkylene-Y and Y, wherein Y is a radical selected from the group consisting of: 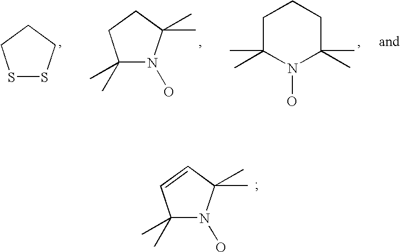
R⁴ may be lower alkyl or H;
R⁵ may be selected from —H, lower alkyl, -alkylene-Y or Y, wherein Y is a radical selected from the group consisting of: 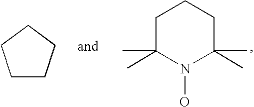
or R⁴ and R⁵ together may form a group selected from the formulae: 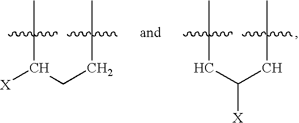
wherein X is selected from H, OH, SH, NH₂, ONO₂, SNO and NONOate.

60. A method according to claim 59, wherein said R³ is selected from

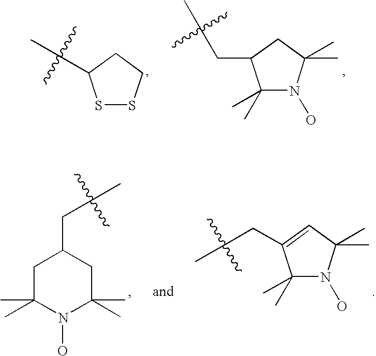

61. A method according to claim 49, wherein said multifunctional ACE-inhibitor has Formula II: 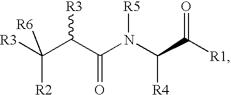
wherein R¹ may be selected from H, OH, NH₂, and alkoxy;
R² may be independently selected from SH and SNO;
R³ may be selected from -alkylene-Y and Y, wherein Y is a radical selected from the group consisting of: 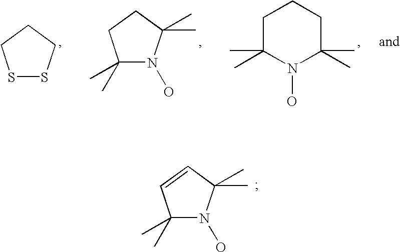
R⁴ may be lower alkyl or H;
R⁵ may be selected from H, lower alkyl, -alkylene-Y and Y, wherein Y is a radical selected from the group consisting of: 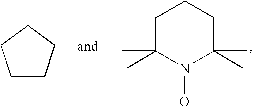
or R⁴ and R⁵ together may form a group selected from the formulae: 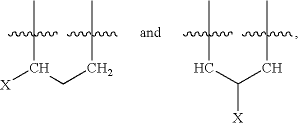
wherein X is selected from H, OH, SH, NH₂, ONO₂, SNO and NONOate; and
R⁶ may be lower alkyl.

62. A method according to claim 49, wherein said multifunctional ACE-inhibitor has Formula III: 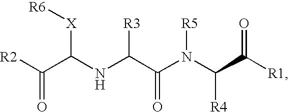
wherein R¹ may be selected from OH, NH₂, alkoxy, and alkyl;
R² may be selected from OH, NH₂, alkoxy, and alkyl;
R³ is lower alkyl; and
R⁶ may be selected from -alkylene-Y and Y, wherein Y is a radical selected from the group consisting of: 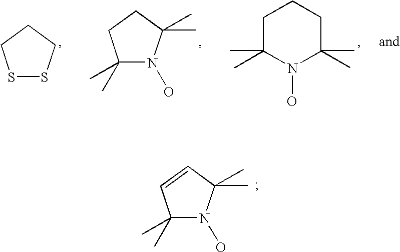
X is (CH₂)ₙ; where n an integer from 0 to 5;
R⁴ is lower alkyl or H;
R⁵ may be selected from H, lower alkyl, -alkylene-Y, and Y, wherein Y is a radical selected from the group consisting of: 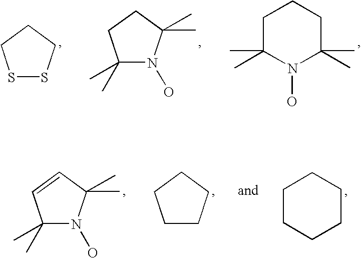
or R⁴ and R⁵ together form a group independently selected from the formulae: 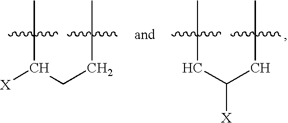
wherein X is selected from H, OH, SH, NH₂, ONO₂, SNO, and NONOate.

63. A method according to claim 62, wherein said R⁶ is selected from

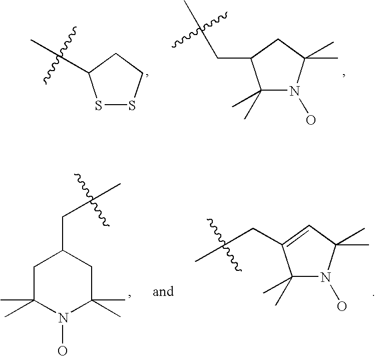

64. A method according to claim 49, wherein said multifunctional ACE-inhibitor has Formula IV: 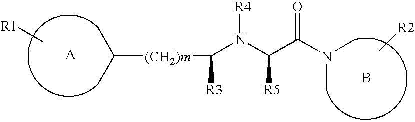
wherein m is an integer from 0 to 5;
A and B are, independently, optionally substituted saturated or unsaturated rings of from 4 to 18 atoms, wherein one or both comprise said ROS scavenger component; and wherein
R¹ and R⁵ are, independently, selected from H, optionally substituted lower alkyl, and (CH₂)ₙX, where n is 0-2 and X is selected from OH, NH₂, SH, ONO, ONO₂, SNO and NONOate;
R² and R³ are, independently, selected from COR⁶ and (CH₂)ₙX,
wherein R⁶ is selected from the group consisting of OH, optionally substituted alkyl, optionally substituted acyl, optionally substituted aryl, optionally substituted heterocyclyl, and optionally substituted cycloalkyl, n is 0-2, and X is selected from OH, NH₂, SH, ONO, ONO₂, SNO, and NONOate;
R⁴ is H or lower alkyl;
A is an optionally substituted saturated or unsaturated ring system of from 4 to 18 atoms; and
B is an optionally substituted, saturated or unsaturated ring system of from 4 to 18 atoms.

65. A method according to claim 64, wherein A is selected from the group consisting of 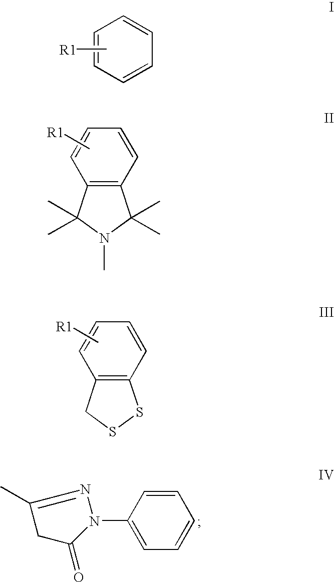
and B is selected from the group consisting of

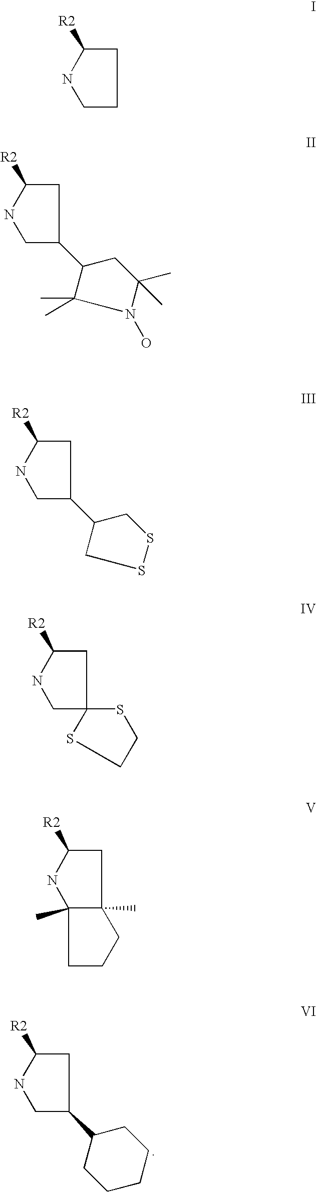

66. A method according to claim 49, wherein said disorder is selected from the group consisting of ischaemic heart disease, angina pectoris, myocardial infarction, congestive heart failure, cardiomyopathy, atherosclerosis, ischaemia-reperfusion tissue injury, peripheral vascular disease, critical limb ischaemia, palpitations, arrhythmia, tachycardia, sinus, thyrotoxicosis, pheochromocytoma, tension, anxiety, alcohol withdrawal, anxiety, migraine, arterial aneurysm, microvascular diseases, hypertension selected from pulmonary-, systemic-, ocular-, obesity-, and pregnancy-induced, impotence, diabetes mellitus, hypercholestemia, Reaven's syndrome, diabetic nephropathy, insulin-resistance and glucose intolerance in diabetes, endothelial dysfunction or oxidative stress-induced diseases, drug or disease induced nephropathy, and esophageal varices.

67. A method according to claim 66, further preventing the occurrence of adverse effects of drugs, the development of tolerance to drugs, or the development of hypersensitivity to drugs.

68. A method according to claim 49, wherein said administering is selected from the group consisting of topical, oral, and parenteral.

69. A method according to claim 49, wherein said administering is selected from the group consisting of suppository, by way of injection, and by way of infusion.

70. A method according to claim 49, wherein said multifunctional ACE-inhibitor is administered by a route selected from intramuscular, intraperitoneal, intravenous, ICV, intracisternal injection or infusion, subcutaneous injection, implant, inhalation spray, nasal, vaginal, rectal, sublingual, and urethral.

71. A method according to claim 49, wherein said mammal is human.

72. A multifunctional ACE-inhibitor comprising
i) an ACE-inhibitor component,
ii) at least one reactive oxygen species (ROS) scavenger component, not identical with said ACE-inhibitor component, and
iii) at least one nitric oxide (NO) donor component, not identical with said ROS scavenger component.

73. A multifunctional ACE-inhibitor according to claim 72 having Formula I: 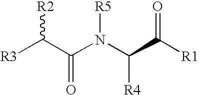
wherein R¹ may be selected from H, OH, NH₂, and alkoxy;
R² may be selected from H and lower alkyl;
R³ may be selected from -alkylene-Y and Y, wherein Y is a radical selected from the group consisting of: 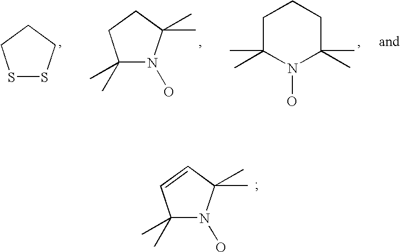
R⁴ may be lower alkyl or H;
R⁵ may be selected from H, lower alkyl, -alkylene-Y or Y, wherein Y is a radical selected from the group consisting of: 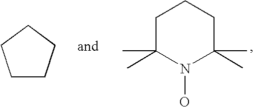
or R⁴ and R⁵ together may form a group selected from the formulae: 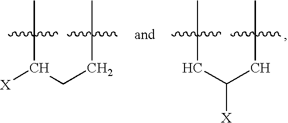
wherein X is selected from H, OH, SH, NH₂, ONO₂, SNO and NONOate.

74. A multifunctional ACE-inhibitor according to claim 72 having Formula II: 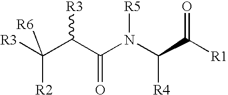
wherein R¹ may be selected from H, OH, NH₂, and alkoxy;
R² may be independently selected from SH and SNO;
R³ may be selected from -alkylene-Y and Y, wherein Y is a radical selected from the group consisting of: 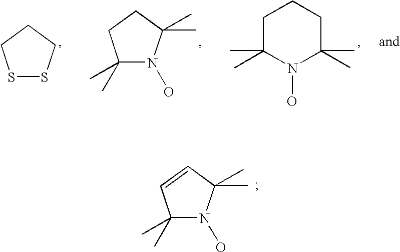
R⁴ may be lower alkyl or H;
R⁵ may be selected from H, lower alkyl, -alkylene-Y and Y, wherein Y is a radical selected from the group consisting of: 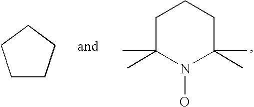
or R⁴ and R⁵ together may form a group selected from the formulae: 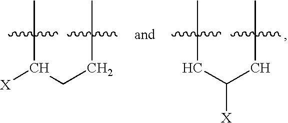
wherein X is selected from H, OH, SH, NH₂, ONO₂, SNO and NONOate; and
R⁶ may be lower alkyl.

75. A multifunctional ACE-inhibitor according to claim 72 having Formula III: 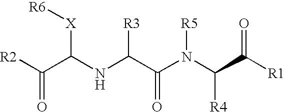
wherein R¹ may be selected from OH, NH₂, alkoxy, and alkyl;
R² may be selected from OH, NH₂, alkoxy, and alkyl;
R³ is lower alkyl; and
R⁶ may be selected from -alkylene-Y and Y, wherein Y is a radical selected from the group consisting of: 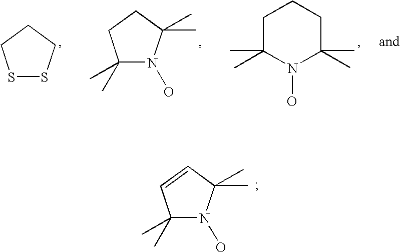
X is (CH₂)ₙ; where n an integer from 0 to 5;
R⁴ is lower alkyl or H;
R⁵ may be selected from H, lower alkyl, -alkylene-Y, and Y, wherein Y is a radical selected from the group consisting of: 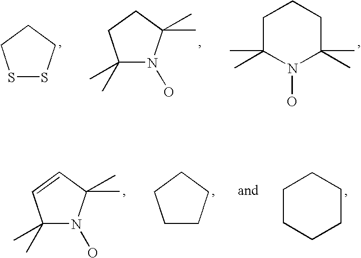
or R⁴ and R⁵ together form a group independently selected from the formulae: 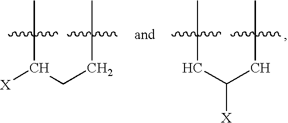
wherein X is selected from H, OH, SH, NH₂, ONO₂, SNO, and NONOate.

76. A multifunctional ACE-inhibitor according to claim 72 having Formula IV: 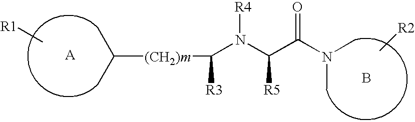
wherein m is an integer from 0 to 5;
A and B are, independently, an optionally substituted saturated or unsaturated rings of from 4 to 18 atoms, wherein one or both comprise said ROS scavenger component; and wherein
R¹ and R⁵ are independently selected from H, optionally substituted lower alkyl, and (CH₂)ₙX, where n is 0-2 and X is selected from OH, NH₂, SH, ONO, ONO₂, SNO and NONOate;
R² and R³ are independently selected from COR⁶ and (CH₂)ₙX, wherein
R⁶ is selected from the group consisting of OH, optionally substituted alkyl, optionally substituted acyl, optionally substituted aryl, optionally substituted heterocyclyl, and optionally substituted cycloalkyl, n is 0-2, and X is selected from OH, NH₂, SH, ONO, ONO₂, SNO, and NONOate;
R⁴ is H or lower alkyl;
A is an optionally substituted saturated or unsaturated ring system of from 4 to 18 atoms; and
B is an optionally substituted, saturated or unsaturated ring system of from 4 to 18 atoms.

77. A pharmaceutical composition comprising an ACE-inhibitor according to claim 72, or a derivative thereof selected from the group consisting of an optical isomer, solvate, and salt.

78. A pharmaceutical composition according to claim 77 further comprising a component selected from the group consisting of a carrier, binding agent, stabilizer, adjuvant, diluent, excipient, surfactant, odorant, and a second pharmaceutically active agent.

79. A kit for administering a multifunctional ACE-inhibitor comprising
i) a dosage amount of at least one compound having a component exhibiting ACE-inhibitor activity and another component exhibiting ROS-scavenging activity;
ii) instructions for use; and
iii) optionally, means for delivery of said compound.
